(19) United States
(12) Patent Application Publication
(10) Pub. No.: US 2007/0092888 A1
(43) Pub. Date: Apr. 26, 2007

(54) Diagnostic markers of hypertension and methods of use thereof

(76) Inventors: Cornelius Diamond, La Jolla, CA (US); Albert Man, La Jolla, CA (US); Troy Bremer, Pana Point, CA (US)

Correspondence Address: William C. Fuess, Suite 2G, 109951 Sorrento Valley Road, San Diego, CA, 92121-1613, US

(21) Appl. No.: 11/435,051

(22) Filed: May 15, 2006

Related U.S. Application Data
(63) Continuation-in-part of application No. 10/948,834, filed on Sep. 22, 2004.
(60) Provisional application No. 60/681,845, filed on May 16, 2005.
(60) Provisional application No. 60/505,606, filed on Sep. 23, 2003.

(51) Int. Cl. C12Q 1/68; G06F 19/00

(52) U.S. Cl. 435/6; 702/20

(57) ABSTRACT

The present invention relates to methods for the diagnosis and evaluation of cardiovascular illness, particularly hypertension treatment. In particular, patient test samples are analyzed for the presence and amount of members of a panel of markers comprising one or more specific markers for hypertension treatment and one or more non-specific markers for hypertension treatment. A variety of markers are disclosed for assembling a panel of markers for such diagnosis and evaluation. Algorithms for determining proper treatment are disclosed. A diagnostic kit for a panel of said markers is disclosed. In various aspects, the invention provides methods for the early detection and differentiation of hypertension treatment. Invention methods provide rapid, sensitive and specific assays that can greatly increase the number of patients that can receive beneficial treatment and therapy, reduce the costs associated with incorrect diagnosis, and provide important information about the prognosis of the patient.

### REFERENCE TO RELATED PREDECESSOR PATENT APPLICATIONS

The present application is descended from, and claims benefit of priority of, U.S. provisional patent application Nos. 60/681,845 filed on May 16, 2005; 60/505,606, filed on May 13, 2005; and 60/556,411 filed on Mar. 24, 2004, the contents of which applications are hereby incorporated herein in their entirety, including all tables, figures, and claims. The present application is a continuation-in-part of, and claims benefit of priority of, U.S. utility patent application Ser. No. 10/948,834, filed on Sep. 22, 2004, which application is itself descended from U.S. Provisional Patent Application No. 60/505,606, filed on Sep. 23, 2003.

### FIELD OF THE INVENTION

The present invention relates to the identification and use of diagnostic markers for hypertension treatment. In various-aspects, the invention relates to methods for the prediction of response to hypertension medication and the development of novel therapies in hypertension treatment and cardiovascular illness.

### BACKGROUND OF THE INVENTION

The following discussion of the background of the invention is merely provided to aid the reader in understanding the invention and is not admitted to describe or constitute prior art to the present invention.

Hypertension is the presence of elevated pressure within the heart and blood vessels that places the patient at increased risk for damage to a number of organs. The risk of complications, such as heart failure, heart attack, kidney failure, blindness, stroke, and death increases as the pressure rises and as tissue is damaged.

It is estimated that as many as 50 million Americans aged 6 and older suffer from hypertension, leading to deaths of 42,565 Americans and contributed to the deaths of about 210,000 in 1997. The total cost to the U.S. economy is estimated to be $19 billion a year as of 1996, growing at a rate of 10% a year (see for instance hftp://www.niddk. nih.gov/health/nutrit/pubs/statobes.htm).

Of the 23.4 million Americans who take anti-hypertensive medication, only 42.9% of these patients are able to control their blood pressure (see for instance Burt V L, Cutler J A, Higgins M, et al: Trends in the prevalence, awareness, treatment, and control of hypertension in the adult US population. Data from the Health Examination Surveys, 1960 to 1991. Hypertension 1995; 26:60-69.).

This failure to control blood pressure costs $964 million annually in general and $467 million among people who are actually being treated for high blood pressure, from a 2000 study. These incremental cost estimates are, in all likelihood, on the low side, as no cost was assigned to death from uncontrolled hypertension (see for instance hftp://www.heartinfo.org/reuters2000/00519elin028.htm).

One of the problems is that the clinical effectiveness of most anti-hypertensive drugs is only in the 40-55% range when used alone. Of those that respond, approximately two-thirds require the highest recommended dose to achieve control (see for instance Materson B J, Reda D J, Cushman W C, et al, for the Department of Veterans Affairs Cooperative Study Group on Antihypertensive Agents: Single drug therapy for hypertension in men. A comparison of six antihypertensive drugs with placebo. N Engl J Med, 1993; 328:914-921.).

Another study that analyzed data on the efficacy of specific drugs in individual patients concluded that 10-59% of patients failed to respond to diuretics, 12-86% failed to respond to β-blockers, some patients exhibited heterogeneous responses to ACE inhibitors and calcium antagonists, and a small percentage of patients even showed an increase in blood pressure (see for instance Neutel J M, Rolf C N, Valentine S N, Li J, Lucus C, Marmorstein B L: Low-dose combination therapy as first line treatment of mild-to-moderate hypertension: the efficacy and safety of bisoprolol/HCTZ versus amlodipine, enalapril, and placebo. Cardiovasc Rev Rep 1996; 71:33-45.). The variation in the individual response to anti-hypertensive drugs may be due to the heterogeneity of the mechanisms underlying hypertension, inter-individual variations of the pharmacokinetics of the drugs, or both.

In addition, much of poor patient compliance leading to treatment failure can be blamed on the side effects caused by anti-hypertensive medication. This factor must be dealt with to achieve blood pressure control.

Most side effects with anti-hypertensive agents are dose dependent. Using smaller doses of various drugs limits dose-dependent side effects. The combination of two complementary agents improves the response rate because more than one physiologic pathway is interrupted, leading to synergistic effects to improve efficacy and avoid the adverse drug reactions associated with higher doses of individual monotherapies. Ideally, one therapy would offset the potential adverse events of the other. Such combinations must address the various factors underlying hypertension in different individuals, including blood volume, vasoconstriction, and the impact of the sympathetic nervous system and the renin-angiotensin system.

Anti-hypertension medications are a primary method for treatment of hypertension. Prescription of anti-hypertensive medication, however, is inexact. Not all patients receiving an anti-hypertensive medication will respond to that treatment. Others may respond, but with serious side effects. The period required to determine the efficacy of treatment response can be both costly and lengthy. Thus a method for rapid identification of appropriate treatment for patients is needed.

Assessing Patient Response to Hypertension Treatment

Responder/non-responder phenotypes of treatment efficacy are determined quantitatively by blood pressure, which first became easy to measure in 1896 when the Italian physician, Riva Rocci, developed what we would now recognize as a conventional mercury sphygmomanometer with a cuff around the arm, which was inflated until the pulsation of the artery could no longer be felt. This gave a very accurate measurement of systolic pressure, although it was subsequently found that it was more accurate if a wider cuff was used. In 1904 Nicolai Korotkoff, a Russian army surgeon, realised that by listening with a stethoscope below the cuff over the artery at the elbow, characteristic sounds were heard at the systolic pressure, but also importantly at the lower pressure (diastolic) when the heart relaxes. It then became very easy to measure both systolic and diastolic pressure accurately with a stethoscope.

Although definitions of hypertension in quantitative measurements of systolic and diastolic blood pressure are continually being modified, usually downwards, the definitions according to the Seventh Joint National Committee on Hypertension (JNC-VII, http://www.nhlbi.nih.gov/quidelines/hypertension; incorporated by reference) is given in table 1.

TABLE 1
Classification and management of blood pressure for adults*

| | | | Initial Drug Therapy | |
|---|---|---|---|---|
| | | | Without Compelling | With Compelling |
| BP Classification | SBP*mmHg | DBP*mmHg | Indication | Indications |
| Normal | <120 | and <80 | No antihypertensive | Drug(s) for compelling |
| | | | drug indicated. | indications.‡ |
| Prehypertension | 120-139 | or 80-89 | | |
| Stage 1 | 140-159 | or 90-99 | Thiazide-type diuretics | Drug(s) for the compelling |
| Hypertension | | | for most. May consider ACEI, | indications.‡ |
| | | | ARB, BB, CCB, or combination. | Other antihypertensive |
| Stage 2 | ≧160 | or ≧100 | Two-drug combination for | drugs (diuretics, ACEI, |
| Hypertension | | | most† (usually | ARB, BB, CCB) as needed. |
| | | | thiazide-type diuretic and | |
| | | | ACEI or ARB or BB or CCB). | |

DBP, diastolic blood pressure; SBP, systolic blood pressure.
Drug abbreviations: ACEI, angiotensin converting enzyme inhibitor; ARB, angiotensin receptor blocker; BB, beta-blocker; CCB, calcium channel blocker.
*Treatment determined by highest BP category.
†Initial combined therapy should be used cautiously in those at risk for orthostatic hypotension.
‡Treat patients with chronic kidney disease or diabetes to BP goal of <130/80 mmHg.

Classification of Patient Response/Non-Response

While the goal of all hypertension therapy is to achieve normotensive status in the patient (Normal blood pressure for an adult is around 120/80 mmHg), sometime this is not achievable and a person skilled in the art of treating hypertension will recognize a patient can still have a ‘response’ to a medication without achieving normotensive status.

Current diagnostic methods for hypertension treatment are basically trial-and-error. A person is given a medication at usually a low dosage, then titrated upwards in dosage over a period of weeks or months. After several months, the person is evaluated again by a physician to determine if the person's hypertension level has changed and/or an adverse event is registered. If it has not changed enough in a positive direction to suit the patient and/or physician, the person is gradually titrated downwards on the first drug and the process repeats itself with another medication. It is not uncommon for a patient to repeat this process over a period of years, all the while suffering physically, emotionally, and financially.

Accordingly, there is a present need in the art for a rapid, sensitive and specific diagnostic assay for hypertension treatment that can differentiate the type of medication and also identify those individuals at risk for adverse events. Such a diagnostic assay would greatly increase the number of patients that can receive beneficial treatment and therapy, and reduce the costs associated with incorrect therapy.

Mechanism of Hypertension

Blood pressure levels are homeostatically maintained through complex networks of many interrelated biochemical, physiologic, and anatomic traits organized to provide redundant systems with counterbalancing pressor and the pressor effects. Despite this underlying complexity, all hypertension can be viewed as a consequence of inappropriate vasoconstriction relative to the concomitant intravascular fluid volume or of overfilling of the arterial vascular bed with excess fluid relative to its capacity. Because many components of the regulatory systems are proteins that may vary in structure, configuration, or quantity because of genetic differences, it is expected that interindividual variation in antihypertensive drug responses are at least in part genetically determined. Logical candidate genes to influence antihypertensive drug responses are those that code for components of the system(s) targeted by the drug or of counter regulatory system(s) opposing an initial drug-induced fall in blood pressure. The only difference between a drug response trait and other genetically influenced traits is that the drug(s) must be administered for the response trait to manifest. Otherwise, the same analytical approaches can be taken to identify and characterize genetic and environmental sources of variation in drug response trait.

Angiotensinogen, of the serine protease inhibitor family, is a cell-secreted plasma protein in the circulation originating predominantly from the liver. It is cleaved by Renin to release a small 10 amino acid protein, Angiotensin I. A positive feedback mechanism exists between Angiotensin II and Angiotensinogen expression.

Angiotensin Converting Enzyme (ACE), adipeptidyl carboxypeptidase, has 2 mechanisms for vasoconstriction. It converts angiotensin I to angiotensin II, which is a potent vasoconstrictor, and it inactivates bradykinin, which is a vasodilator. ACE contains one functional site for cleaving terminal dipeptides. There are different classes of ACE inhibitors. Non-peptide inhibitors chealate Zinc and heavy metal ions needed for enzymatic activity, and creates a catalytically defective enzyme. A second class of inhibitors are peptides that interact with ACE similarly to endogenous substrates. Most medications are in this class.

Angiotensin II receptors bind free angiotensin II and initiate the biochemical signal pathways that lead to many physiological effects downstream effects. It is a member of the superfamily of G protein-coupled receptors that have seven transmembrane regions.

Calcium channel blockers (CCBs), which include both dihydropyridines such as nifedipine and amlodipine and non-dihydropyridines (verapamil and diltiazem), are among the most widely prescribed agents for the management of essential hypertension. Several large outcome risk trials and comprehensive meta-analyses have found that CCBs reduce the cardiovascular morbidity and mortality associated with uncontrolled hypertension, including stroke. CCBs, however, appear less effective than angiotensin-converting enzyme inhibitors and diuretics for preventing heart failure and myocardial infarction. CCBs are among the agents listed as potential first-line therapy, either alone or in combination with other agents in hypertension management guidelines. Furthermore, CCBs are suitable for add-on therapy in combination with diuretics, angiotensin-converting enzyme inhibitors, and angiotensin-II receptor blockers.

Ca²⁺ plays a unique role among ions by acting as an intracellular messenger. Ca²⁺-binding proteins sense increases in Ca²⁺ concentration to trigger cellular processes such as muscle contraction, neurotransmitter and hormone release, changes in gene transcription, and changes in neuronal excitability. One function of voltage-dependent calcium channels is to link electrical excitability (e.g., action potential) to cellular action; however, the influx of Ca²⁺ through these channels also depolarizes the plasma membrane in the same manner as Na+. Thus, a second function is to depolarize the cell membrane to generate action potentials (Bertolino M, Llinas R R. 1992. The central role of voltage-activated and receptor-operated calcium channels in neuronal cells. Annu Rev Pharmacol Toxicol 32: 399-421.). Whether the Ca²⁺ influx is involved in action potential generation, activation of intracellular signaling pathways, or both, hyperactivity of the voltage-dependent calcium channels can lead to pathophysiology.

Like most ion channels, voltage-dependent calcium channels are composed of various subunits. A single polypeptide (α₁ subunit) forms the basic channel structure and contains the amino acids that form the pore, voltage sensor, activation gate, and inactivation gate (Catterall Wash. 2000. Structure and regulation of voltage-gated Ca2+ channels. Annu Rev Cell Dev Biol 16: 521-555.). The other channel components, called auxiliary subunits, are the β, α₂δ and Ysubunits. These subunits seem to modulate function of the α₁ subunit (Arikkath J, Campbell K P. 2003. Auxiliary subunits: essential components of the voltage-gated calcium channel complex. Curr Opin Neurobiol 13: 298-307.).

Calcium channel nomenclature is shown in Table II. An additional nomenclature that is not in the table separates calcium channels by activation voltage range, high voltage-activated (HVA) and low voltage-activate (LVA). All the CaV3 channels (T-type) are LVA, whereas the CaV1 and CaV2 channels are HVA. The primary reason they were separated initially was that blockers that could differentiate some of the channels were identified. It was determined later that multiple genes generate the different pharmacologic classes; however, no blockers have yet been identified that can unambiguously separate the four different CaV1 (L-type) or the three different CaV3 (T-type) channels.

TABLE II
Calcium Channel Nomenclature and Blockers*

| Pharmacology | mRNA | Gene | Common blockers |
|---|---|---|---|
| L-type | α_{1S} | Ca_{V}1.1 | Nifedipine, amlodipine |
| (skeletal muscle) | | | |
| L-type | α_{1C} | Ca_{V}1.2 | Nifedipine, amlodipine |
| (cardiac muscle) | | | |
| L-type (endocrine) | α_{1D} | Ca_{V}1.3 | Nifedipine, amlodipine |
| L-type (retina) | α_{1F} | Ca_{V}1.4 | Nifedipine, amlodipine |
| P/Q-type | α_{1A} | Ca_{V}2.1 | ω-AgaIVA, ω-conotoxin MVIIC |
| N-type | α_{1B} | Ca_{V}2.2 | ω-Conotoxin GVIA, ziconotide |
| R-type | α_{1E} | Ca_{V}2.3 | SNX482^{a}, Ni²⁺ |
| T-type | α_{1G} | Ca_{V}3.1 | Mibefradil, ethosuxamide, Ni²⁺ |
| T-type | α_{1H} | Ca_{V}3.2 | Mibefradil, ethosuxamide, Ni²⁺ |
| T-type | α_{1I} | Ca_{V}3.3 | Mibefradil, ethosuxamide, Ni²⁺ |

*Different nomenclatures are based on blocker sensitivity (pharmacology), the order in which the mRNA was cloned (A-I), and gene homology (Ertel et al., [2000]). Blockers listed are ones that are used commonly, but this is not a complete list.
^{a}Indicates that Ca_{V}2.3 current is partially blocked.

Certain channel types are localized to specific tissues, whereas others are expressed more generally. CaV1.1 is expressed exclusively in skeletal muscle and links membrane potential changes to calcium release from the sarcoplasmic reticulum (SR). CaV2 channels are found throughout the cardiovascular and nervous systems and are the target of antihypertensive calcium channel blockers. In the heart, they are found in the T-tubules associated closely with SR Ryanodine receptor/Ca²⁺-release channels (RyR), where Ca²⁺ influx through CaV1.2 channels activates RyR to release Ca²⁺ via Ca²⁺-induced Ca²⁺ release (CICR) (Bers D M, Perez-Reyes E. 1999. Ca channels in cardiac myocytes: structure and function in Ca influx and intracellular Ca release. Cardiovasc Res 42: 339-360.). CaV1.2 channels are activated generally by action potentials, but arterial smooth muscle seems to have sufficiently depolarized steady-state membrane potential to constitutively activate smooth muscle CaV1.2 channels (Nelson M T, Patlak J B, Worley J F, Standen N B. 1990. Calcium channels, potassium channels, and voltage dependence of arterial smooth muscle tone. Am J Physiol 259: 3-18.). CaV1.2 channels have brief open times relative to CaV2 channels, so they generate less Ca²⁺ flux/opening. This small Ca²⁺ influx would be a problem for cardiac muscle contraction if it were not for CICR as described above by Bers and Perez-Reyes, [1999]. Although the CaV1.2-RyR interaction is tuned precisely for cardiac muscle contraction, the Ca²⁺ delivered by CaV1.2 channels may be too small to trigger fast neurotransmifter release, which is dominated by CaV2 channels (Turner T J, Adams M E, Dunlap K. 1993. Multiple Ca2+ channel types coexist to regulate synaptosomal neurotransmitter release. Proc Natl Acad Sci USA 90: 9518-9522.). CaV1.3 channels are associated generally with neuroendocrine cells, such as pituitary cells and pancreatic islet cells (Safa P, Boulter J, Hales T G. 2001. Functional properties of CaV1.3 (alpha 1D) L-type Ca2+ channel splice variants expressed by rat brain and neuroendocrine GH3 cells. J Biol Chem 276: 38727-38737.), but are found also in the cardiac atrioventricular node (Zhang et al., 2002b. Functional roles of Cav1.3 (alpha)1D calcium channel in sinoatrial nodes: insight gained using gene-targeted null mutant mice. Circ Res 90: 981-987.). These channels have been reported to be crucial for delivering the Ca²⁺ needed to trigger insulin release and their deletion is associated with cardiac arrhythmia. CaV2 channels are primarily neuronal and are found in the soma, dendrites, and nerve terminals. CaV2.1 (P/Q-type) and CaV2.2 (N-type) channels seem to be the dominant calcium channels involved in synaptic transmission (Turner et al., [ibid]), although CaV2.3 channels may also play a role (Gasparini et al., Presynaptic R-type calcium channels contribute to fast excitatory synaptic transmission in the rat hippocampus. J Neurosci 21: 8715-8721.). Action potentials are the primary physiologic activator of these channels, which remain open during much of the repolarization phase of the action potential where electrochemical forces can drive Ca²⁺ into the cell (Park and Dunlap, [1998] Dynamic regulation of calcium influx by G-proteins, action potential waveform, and neuronal firing frequency. J Neurosci 18: 6757-6766.).

The calcium channel blockers prescribed most frequently target CaV1 channels, specifically CaV1.2 channels that are highly expressed in cardiac and smooth muscle. The three most commonly prescribed drug classes are phenylalkylamines (PAA; e.g., verapamil), benzothiazepines (e.g., diltiazem), and dihydropyridines (DHP; e.g., nifedipine or amlodipine). These drugs are prescribed for a wide range of pathologies including hypertension, angina, congestive heart failure, and premature labor. They have a low incidence of side effects (Messerli, [200] Calcium antagonists in hypertension: from hemodynamics to outcomes. Am J Hypertens 15: 94-97.), which is particularly important in pregnancy. Each drug class binds to distinct sites on the α₁ subunit that seem to mediate unique blocking mechanisms (Striessnig et al., [1998] Structural basis of drug binding to L Ca2+ channels. Trends Pharmacol Sci 19: 108-115.). The block by PAAs is use dependent, which is explained by a requirement for channel opening before block can occur. Thus, the activation gate may obscure the PAA binding site to prevent block of closed channels (Striessnig et al., [1998, ibid]). On the other hand, DHPs show little or no use-dependent block (Sanguinetti and Kass, [1984], Voltage-dependent block of calcium channel current in the calf cardiac Purkinje fiber by dihydropyridine calcium channel antagonists. Circ Res 55: 336-348.). This class of blockers seems to inhibit by binding preferentially to the inactivated state (Bean, [1984], Nitrendipine block of cardiac calcium channels: high-affinity binding to the inactivated state. Proc Natl Acad Sci USA 81: 6388-6392.). Because inactivation is favored by depolarization, the affinity of CaV1 channels for DHPs is increased substantially by membrane depolarization (Bean, [1984, ibid]). The different blocking mechanisms of these two drug classes dictate different clinical uses. The use-dependent block by PAAs make them better for treatment of arrhythmias, whereas the depolarized resting potential of vascular smooth muscle seems to be one reason DHPs preferentially decrease peripheral resistance (i.e., lower blood pressure) without affecting cardiac contraction (Hernandez-Hernandez et al., [2002], Update on the use of calcium antagonists on hypertension. J Hum Hypertens 16(Suppl): 114-117.).

In clinical practice, the β-adrenergic antagonists are an extremely important class of drugs due to their high prevalence of use. Many have been synthesized and are commonly used systemically in the treatment of conditions including hypertension, cardiac arrhythmia, angina pectoris, and acute anxiety, and topically for open angle glaucoma. With respect to their clinical utility, the beta-blockers are normally distinguished based on their selectivity for beta-receptors. Beta receptors in the heart are mediators of ion transport and homeostasis and, with sustained stimulation, can cause adverse effects on the timing of myocardial muscle contraction. β₁- and β₂-adrenergic receptors also mediate the electrophysiologic effects of catecholamines. The nonselective beta-blockers, including propranolol, oxprenolol, pindolol, nadolol, timolol and labetalol, each antagonize both β₁- and β₂-adrenergic receptors. For the selective antagonists, including metoprolol, atenolol, esmolol, and acebutolol, each has much greater binding affinity for the b₁ adrenergic receptor. The selective beta-blockers are normally indicated for patients in whom β₂-receptor antagonism might be associated with an increased risk of adverse effects. Such patients include those with asthma or diabetes, or patients with peripheral vascular disease or Raynaud's disease.

A common feature in the chemical structure of beta-blockers is that there is at least one aromatic ring structure attached to a side alkyl chain possessing a secondary hydroxyl and amine functional group. Each of the available beta-blockers has one or more chiral centers in its structure, and in all cases, at least one of the chiral carbon atoms residing in the alkyl side chain is directly attached to a hydroxyl group. Except for timolol, which is marketed as S-enantiomer, each of the beta-blockers with one chiral center (e.g., propranolol, metoprolol, atenolol, esmolol, pindolol, and acebutolol) is marketed as a racemate consisting of two enantiomers. Additionally, labetalol, which has two chiral centers, is marketed as a racemate consisting of four isomers. Nadolol is a drug has three chiral centers in its structure. However, the two ring hydroxyl groups are in the cis orientation, allowing for only four isomers. More detail is given in U.S. Pat. No. 4,337,207.

Currently, β-blockers are recommended firstline agents in the management of hypertension. However, marked variability in response exists, with adequate blood pressure control failing to be achieved with β-blocker monotherapy in 30% to 60% of patients (see for instance Materson B J, et al. Single-drug therapy for hypertension in men. A comparison of six antihypertensive agents with placebo. The Department of Veterans Affairs Cooperative Study Group on Antihypertensive Agents. N Engl J Med 1993; 328:914-21.). The factors responsible for this interpatient variability are not well understood; however, genetic differences in the β₁-adrenergic receptor (b₁AR) may contribute. Two common polymorphisms occur in the gene that encodes the β1AR. Both are single-nucleotide polymorphisms (SNPs), with an A3G substitution occurring at nucleotide 145, resulting in a Ser to Gly substitution at codon 49, and a G3C substitution at nucleotide 1165, resulting in an Arg to Gly substitution at codon 389. There are racial differences in allele frequencies for both polymorphisms, with Gly49 frequencies of 0.15 and 0.29 and Gly389 frequencies of 0.27 and 0.42 in white subjects and black subjects, respectively. (see for instance Maqbool A, Hall A S, Ball S G, Balmforth A J. Common polymorphisms of β₁-adrenoceptor: identification and rapid screening assay. Lancet 1999; 353:897.) The codon 49 polymorphism is located in the extracellular amino terminus. The codon 389 polymorphism occurs in an intracellular domain proximal to the seventh transmembrane-spanning region of the receptor. This area is highly conserved and important in G-protein coupling.

In recent years, the search for a single gene responsible for major depressive disorder has given way to the understanding that multiple gene variants, acting together with yet unknown environmental risk factors or developmental events, interact in a complex system to account for its expression phenotype. In accordance, treatments that successfully alleviate hypertension symptoms are likely to act on multiple gene products of the angiotensin-renin pathway and others.

Research has indicated that characteristics such as age, gender, ethnicity, weight, diagnosis, and diet affect both the pharmacokinetics and pharmacodynamics of hypertension medication ( see for instance Williams B., Kim, J., Cardiovascular drug therapy in the elderly: theoretical and practical considerations. Drugs Aging. 2003; 20(6):445-63.; Ethn. Dis. 1998 Winter; 8(1):98-102. Calcium antagonists—pharmacologic considerations. Prisant L M.; Wassertheil-Smoller, S; Anderson, G; Psaty, B; Black, H; et al. Hypertension and Its Treatment in Postmenopausal Women: Baseline Data from the Women's Health Initiative, Hypertension. 2000 36:780; The rationale and design of the AASK cohort study. Appel L J et al. J Am Soc Nephrol. 2003 July; 14(7 Suppl 2):S166-72; Population analyses of sustained-release verapamil in patients: effects of sex, race, and smoking. Kang D, Verotta D, Krecic-Shepard M E, Modi N B, Gupta S K, Schwartz J B. Clin. Pharmacol. Ther. 2003 January; 73(1):31-40.). However, no method currently exists for incorporating these variables into a predictive algorithm for prescribing medication.

Recently, attention has focused on the identification of Single Nucleotide Polymorphisms, (hereafter SNPs) as factors that specifically influence drug action or act as markers for alleles of genes that influence drug action in hypertension ( see for instance Sethi A A, Nordestgaard B G, Tybjaerg-Hansen A. Angiotensinogen gene polymorphism, plasma angiotensinogen, and risk of hypertension and ischemic heart disease: a meta-analysis. Arterioscler Thromb Vasc Biol. 2003 Jul. 1; 23(7):1269-75. Epub 2003 Jun. 12.; Bengtsson K, Melander O, Orho-Melander M, Lindblad U, Ranstam J, Rastam L, Groop L. Polymorphism in the beta(1)-adrenergic receptor gene and hypertension. Circulation. 2001 Jul. 10; 104(2):187-90.). However, due to reasons described below, these single SNP variants have been shown to have little or no clinically acceptable and/or statistically significant effect by themselves.

As an independent variable, either a SNP or a patient characteristic is unlikely, itself, to indicate a responder phenotype with acceptable confidence—a direct causal effect on phenotype is rare. However, understanding the complex interactions that result in a response phenotype for more than a small number of variables are not realistic without comprehensive analysis technology. This patent will show how to use such analysis algorithms that have the ability to extract meaningful information from complex interactions occurring between multiple variables.

To date, SNPs and various proteins have not been used in combination as markers of hypertension. The current state of the art is single-marker tests which have little or no predictive value in hypertension response over large populations. Myraid Genetics Incorporated, of Salt Lake City, Utah has in the past offered a test for the M235T gene variant in relation to cardiovascular prognosis, including ACE inhibitor response. However, this has not been shown to be of relevance for hypertension in recent studies (see for instance the 1000-plus patient studies Poch E. et al., Genetic polymorphisms of the renin-angiotensin system and essential hypertension, Med Clin (Barc). 2002 Apr. 27; 118(15):575-9.; and Matsubara M et al., T+31C polymorphism (M235T) of the angiotensinogen gene and home blood pressure in the Japanese general population: the Ohasama Study. Hypertens Res. 2003 January; 26(1):47-52.). In addition, Myraid Genetics Incorporated has applied for a patent (U.S. patent Office Ser. No. 10/331,192 filed Dec. 27, 2002) on relating A145G genetic variation in the human beta-1 adrenergic receptor gene to predicting human hypertension medication response. This as well has been shown (see for instance Bengtsson K, Melander O, Orho-Melander M, Lindblad U, Ranstam J, Rastam L, Groop L. Polymorphism in the beta-(1)- adrenergic receptor gene and hypertension. Circulation. 2001 Jul. 10; 104(2):187-90.) to have little or no direct effect on hypertension response. It should now be clear that to diagnosis or determine treatment outcome in a complex disease such as hypertension or cardiovascular disease it is necessary to use multi-factorial genetic and/or proteomic markers and/or inclusive with environment and psysiological variables in combination. The present invention provides for methods for doing exactly this. Preferred markers of the invention can aid in the treatment, diagnosis, differentiation, and prognosis of patients with hypertension, cardiovascular disease, and stroke.

### SUMMARY OF THE INVENTION

In another preferred application of the instant invention relates to the identification and use of diagnostic and/or prognostic markers for anti-hypertensives, the ACE Inhibitors captopril, benazepril, enalapril, enalaprilat, fosinopril, lisinopril, quinapril, ramipril, and trandolapril or others with similar molecular mechanisms; the Calcium Channel Blockers nifedipine, verapamil, nicardipine, diltiazem, isradipine, amlodipine, nimodipine, felodipine, nisoldipine, bepridil or others with similar molecular mechanisms; and the Beta-Blockers atenolol, metoprolol, propranolol, timolol, nadolol, acebutolol, pindolol, sotalol, labetalol, oxprenolol or others with similar molecular mechanisms. The methods and compositions described herein can meet the need in the art for a rapid, sensitive and specific diagnostic assay to be used to facilitate the treatment of hypertension patients and the development of additional diagnostic indicators. Moreover, the methods and compositions of the instant invention can also be used in diagnosis, differentiation and prognosis of various forms of cardiovascular disorders as well as cardiovascular drug discovery.

In yet another aspect, the instant invention features methods of diagnosing hypertension by analyzing a test sample obtained from a patient for the presence or amount of one or more SNPs associated with genes in the adsorption, distribution, receptor or effector biochemical pathways of anti-hypertension medications. These methods can include identifying one or more SNPs, the presence or amount of which is associated with the treatment, diagnosis, prognosis, or differentiation of hypertension. Once such SNP(s) are identified, the pattern of such SNPs in a patient sample can be measured. In certain embodiments, these markers can be compared to a diagnostic level determined by an algorithm that is associated with the treatment, diagnosis, prognosis, or differentiation of hypertension. By correlating the patient pattern to the diagnostic pattern, the presence or absence of hypertension, and the probability of treatment outcomes in a patient may be rapidly and accurately determined.

For purposes of the following discussion, the methods described as applicable to the treatment outcome and diagnosis of hypertension treatment generally may be considered applicable to the treatment outcome and diagnosis of cardiac failure and other cardiovascular diseases such as stroke and atherosclerosis.

In certain embodiments, a plurality of SNPs are combined to increase the predictive value of the analysis in comparison to that obtained from the markers individually or in smaller groups. Preferably, one or more specific markers for hypertension treatment can be combined with one or more non-specific markers for hypertension treatment to enhance the predictive value of the described methods.

In certain embodiments, a diagnostic or prognostic indicator is correlated to a condition or disease by merely its presence or absence. In other embodiments, an algorithm is needed to relate the pattern of markers to a desired prediction outcome in the patient. A preferred algorithmic technique for relating markers of the present invention is a linear regression technique, a nonlinear regression technique, an ANOVA technique, a neural network technique, a genetic algorithm technique, a support vector machine technique, a greedy algorithm technique, a tree algorithm technique, a kernel-based technique, and a Bayesian technique. The skilled artisan will recognize the word “technique” refers to a process in which a predictor is built by using patient exemplar pairs of markers and phenotypes, and then refining such predictor algorithm in an iterative process by testing a version of the algorithm on unseen data and making changes to mathematical coefficients of such algorithm in such a way to increase the accuracy and specificity of the predictor algorithm.

In other embodiments, the invention relates to methods for determining a treatment regimen for use in a patient diagnosed with hypertension, particularly for ACE Inhibitors. The methods preferably comprise determining a level of one or more diagnostic or prognostic markers as described herein, and using the markers to determine a diagnosis for a patient. One or more treatment regimens that improve the patient's prognosis by reducing the increased disposition for an adverse outcome associated with the diagnosis can then be used to treat the patient. Such methods may also be used to screen pharmacological compounds for agents capable of improving the patient's prognosis as above.

In yet another embodiment, multiple determination of one or more diagnostic or prognostic markers can be made, and a temporal change in the marker can be used to monitor the efficacy of appropriate therapies. In such an embodiment, one might expect to see a decrease or an increase in the marker(s) over time during the course of effective therapy.

In yet other embodiments, multiple determination of one or more diagnostic or prognostic markers can be made, and a temporal change in the marker can be used to determine a diagnosis or prognosis. For example, a diagnostic indicator may be determined at an initial time, and again at a second time. In such embodiments, an increase in the marker from the initial time to the second time may be diagnostic of a particular type of hypertension, such as treatment-resistant hypertension, or a given prognosis. Likewise, a decrease in the marker from the initial time to the second time may be indicative of a particular type of hypertension, or a given prognosis. Furthermore, the degree of change of one or more markers may be related to the severity of the disease and future adverse events.

In a further aspect, the invention relates to kits for determining the diagnosis or prognosis of a patient. These kits preferably comprise devices and reagents for measuring one or more SNP patterns or marker levels in a patient sample, and instructions for performing the assay. Optionally, the kits may contain computer algorithms or other means for converting SNP patterns or marker level(s) to a prognosis. Such kits preferably contain sufficient reagents to perform one or more such determinations.

### DETAILED DESCRIPTION OF THE INVENTION

In accordance with the instant invention, there are provided methods and compositions for the identification and use of markers that are associated with the diagnosis, prognosis, or differentiation of hypertension in a patient. Such markers can be used in diagnosing and treating a patient and/or to monitor the course of a treatment regimen; and for screening compounds and pharmaceutical compositions that might provide a benefit in treating or preventing such conditions.

Definitions

Before describing this application of the instant invention in greater detail, the following definitions are set forth to illustrate and define the meaning and scope of the terms used to describe the invention herein.

The terms “cardiovascular disease and anti-hypertensives” relate to the diseases of hypertension, cardiac failure, stroke, other cardiovascular or renal disorders and the pharmaceutical agents used to treat them, respectively. One skilled in the art will recognize these terms, which are described in “The Merck Manual of Diagnosis and Therapy” Seventeenth Edition, 1999, Mark H. Beers, and Robert Berkow, editors, chapters 197-213, incorporated by reference only. In various aspects, the invention relates to materials and procedures for identifying markers that are associated with the diagnosis, prognosis, or differentiation of hypertension treatment in a patient; to using such markers in diagnosing and treating a patient and/or to monitor the course of a treatment regimen; and for screening compounds and pharmaceutical compositions that might provide a benefit in treating or preventing such conditions.

The terms “genetic variant,” “mutation,” “nucleotide variant,” and “nucleotide substitution” are used herein interchangeably to refer to nucleotide changes in a reference nucleotide sequence of a particular gene.

The term “gene”, when used herein, encompasses genomic, mRNA and cDNA sequences encoding the gene protein, including the untranslated regulatory regions of the genomic DNA.

The term “genotype” as used herein means the nucleotide characters at a particular nucleotide variant marker (or locus) in either one allele or both alleles of a gene (or a particular chromosome region). A genotype can be homozygous or heterozygous. Accordingly, “genotyping” means determining the genotype, that is, the nucleotide(s) at a particular gene locus.

As used herein, the terms “amino acid variant,” “amino acid mutation,” and “amino acid substitution” are used herein interchangeably to refer to amino acid changes to a reference protein sequence resulting from a genetic variant or a mutation to the reference gene sequence encoding the reference protein.

The term, “reference sequence” refers to a polynucleotide or polypeptide sequence known in the art, including those disclosed in publicly accessible databases, e.g., GenBank, or a newly identified gene or protein sequence, used simply as a reference with respect to the genetic variant or amino acid variant provided in the present invention.

The term “allele” or “gene allele” is used herein to refer generally to a gene having a reference sequence or a gene containing a specific genetic variant.

The term “locus” refers to a specific position or site in a gene sequence or protein sequence. Thus, there may be one or more contiguous nucleotides in a particular gene locus, or one or more amino acids at a particular locus in a polypeptide. Moreover, “locus” may also be used to refer to a particular position in a gene sequence where one or more nucleotides have been deleted, inserted, or inverted.

As used herein, the terms “polypeptide,” “protein,” and “peptide” are used interchangeably to refer to amino acid chains in which the amino acid residues are linked by covalent peptide bonds. The amino acid chains can be of any length of at least two amino acids, including full-length proteins. Unless otherwise specified, the terms “polypeptide,” “protein,” and “peptide” also encompass various modified forms thereof, including but not limited to glycosylated forms, phosphorylated forms, etc. This term also does not specify or exclude post-translation modifications of polypeptides. For example, polypeptides that include the covalent attachment of glycosyl groups, acetyl groups, phosphate groups, lipid groups and the like are expressly encompassed by the term polypeptide. Also included within the definition are polypeptides which contain one or more analogs of an amino acid (including, for example, non-naturally occurring amino acids, amino acids which only occur naturally in an unrelated biological system, modified amino acids from mammalian systems, etc.), polypeptides with substituted linkages, as well as other modifications known in the art, both naturally occurring and non-naturally occurring.

The terms “primer,” “probe,” and “oligonucleotide” may be used herein interchangeably to refer to a relatively short nucleic acid fragment or sequence. They can be DNA, RNA, or a hybrid thereof, or a chemically modified analog or derivatives thereof. Typically, they are single stranded. However, they can also be double-stranded having two complementing strands which can be separated apart by denaturation. Normally, they have a length of from about 8 nucleotides, and more preferably about 18 to about 50 nucleotides. They can be labeled with detectable markers or modified in any conventional manners for various molecular biological applications.

A “promoter” refers to a DNA sequence recognized by the synthetic machinery of the cell required to initiate the specific transcription of a gene.

The term “primer” denotes a specific oligonucleotide sequence which is complementary to a target nucleotide sequence and used to hybridize to the target nucleotide sequence. A primer serves as an initiation point for nucleotide polymerization catalyzed by either DNA polymerase, RNA polymerase or reverse transcriptase.

The term “probe” denotes a defined nucleic acid segment (or nucleotide analog segment, e.g., polynucleotide) which can be used to identify a specific polynucleotide sequence present in samples, said nucleic acid segment comprising a nucleotide sequence complementary to the specific polynucleotide sequence to be identified.

The location of nucleotides in a polynucleotide with respect to the center of the polynucleotide are described herein in the following manner. When a polynucleotide has an odd number of nucleotides, the nucleotide at an equal distance from the 3′ and 5′ ends of the polynucleotide is considered to be “at the center” of the polynucleotide, and any nucleotide immediately adjacent to the nucleotide at the center, or the nucleotide at the center itself is considered to be “within 1 nucleotide of the center.” With an odd number of nucleotides in a polynucleotide any of the five nucleotides positions in the middle of the polynucleotide would be considered to be within 2 nucleotides of the center, and so on. When a polynucleotide has an even number of nucleotides, there would be a bond and not a nucleotide at the center of the polynucleotide. Thus, either of the two central nucleotides would be considered to be “within 1 nucleotide of the center” and any of the four nucleotides in the middle of the polynucleotide would be considered to be “within 2 nucleotides of the center”, and so on. For polymorphisms which involve the substitution, insertion or deletion of 1 or more nucleotides, the polymorphism, allele or biallelic marker is “at the center” of a polynucleotide if the difference between the distance from the substituted, inserted, or deleted polynucleotides of the polymorphism and the 3′ end of the polynucleotide, and the distance from the substituted, inserted, or deleted polynucleotides of the polymorphism and the 5′ end of the polynucleotide is zero or one nucleotide. If this difference is 0 to 3, then the polymorphism is considered to be “within 1 nucleotide of the center.” If the difference is 0 to 5, the polymorphism is considered to be “within 2 nucleotides of the center.” If the difference is 0 to 7, the polymorphism is considered to be “within 3 nucleotides of the center,” and so on.

The term “upstream” is used herein to refer to a location which is toward the 5′ end of the polynucleotide from a specific reference point.

The terms “base paired” and “Watson & Crick base paired” are used interchangeably herein to refer to nucleotides which can be hydrogen bonded to one another by virtue of their sequence identities in a manner like that found in double-helical DNA with thymine or uracil residues linked to adenine residues by two hydrogen bonds and cytosine and guanine residues linked by three hydrogen bonds (See Stryer, L., Biochemistry, 4.sup.th edition, 1995).

The terms “complementary” or “complement thereof” are used herein to refer to the sequences of polynucleotides that are capable of forming Watson & Crick base pairing with another specified polynucleotide throughout the entirety of the complementary region. For the purpose of the present invention, a first polynucleotide is deemed to be complementary to a second polynucleotide when each base in the first polynucleotide is paired with its complementary base. Complementary bases are, generally, A and T (or A and U), or C and G. “Complement” is used herein as a synonym from “complementary polynucleotide”, “complementary nucleic acid” and “complementary nucleotide sequence”. These terms are applied to pairs of polynucleotides based solely upon their sequences and not any particular set of conditions under which the two polynucleotides would actually bind.

The term “isolated” requires that the material be removed from its original environment (e. g., the natural environment if it is naturally occurring). For example, a naturally-occurring polynucleotide or polypeptide present in a living animal is not isolated, but the same polynucleotide or DNA or polypeptide, separated from some or all of the coexisting materials in the natural system, is isolated. Such polynucleotide could be part of a vector and/or such polynucleotide or polypeptide could be part of a composition, and still be isolated in that the vector or composition is not part of its natural environment.

Specifically excluded from the definition of “isolated” are: naturally-occurring chromosomes (such as chromosome spreads), artificial chromosome libraries, genomic libraries, and cDNA libraries that exist either as an in vitro nucleic acid preparation or as a transfected/transformed host cell preparation, wherein the host cells are either an in vitro heterogeneous preparation or plated as a heterogeneous population of single colonies. Also specifically excluded are the above libraries wherein a specified 5′ EST makes up less than 5% of the number of nucleic acid inserts in the vector molecules. Further specifically excluded are whole cell genomic DNA or whole cell RNA preparations (including said whole cell preparations which are mechanically sheared or enzymaticly digested). Further specifically excluded are the above whole cell preparations as either an in vitro preparation or as a heterogeneous mixture separated by electrophoresis (including blot transfers of the same) wherein the polynucleotide of the invention has not further been separated from the heterologous polynucleotides in the electrophoresis medium (e.g., further separating by excising a single band from a heterogeneous band population in an agarose gel or nylon blot).

The term “purified” does not require absolute purity; rather, it is intended as a relative definition. Purification of starting material or natural material to at least one order of magnitude, preferably two or three orders, and more preferably four or five orders of magnitude is expressly contemplated. As an example, purification from 0.1% concentration to 10% concentration is two orders of magnitude.

The term “purified polynucleotide” or “purified polynucleotide vector” is used herein to describe a polynucleotide or polynucleotide vector of the invention which has been separated from other compounds including, but not limited to other nucleic acids, carbohydrates, lipids and proteins (such as the enzymes used in the synthesis of the polynucleotide), or the separation of covalently closed polynucleotides from linear polynucleotides. A polynucleotide is substantially pure when at least about 50%, preferably 60 to 75% of a sample exhibits a single polynucleotide sequence and conformation (linear versus covalently closed). A substantially pure polynucleotide typically comprises about 50%, preferably 60 to 90% weight/weight of a nucleic acid sample, more usually about 95%, and preferably is over about 99% pure. Polynucleotide purity or homogeneity is indicated by a number of means well known in the art, such as agarose or polyacrylamide gel electrophoresis of a sample, followed by visualizing a single polynucleotide band upon staining the gel. For certain purposes higher resolution can be provided by using HPLC or other means well known in the art.

The invention also concerns gene-related biallelic markers. As used herein the term “gene-related biallelic marker” relates to a set of biallelic markers in linkage disequilibrium with said named gene.

The terms “hypertension” and “hypertensive” used herein refer to symptoms related to undesirably high levels of blood pressure. Individuals said to have “symptoms related to hypertension ” have blood pressure levels at an undesirably high level. For example, an individual with a diastolic blood pressure above 89 mmHg and a systolic blood pressure above 139 mmHg, is considered to have an undesirably high level of blood pressure by the medical community.

“Antihypertensive” treatment and “treating hypertension” as used herein refer to treatment intended to reduce diastolic and/or systolic blood pressure from an undesirably high level (i.e., a level that is considered a disease or disorder under conventional medical standards, or a level that is desired to be reduced for any reason). Individuals with only temporary periods of hypertension—wherein their blood pressure levels only temporarily exceed levels which become undesirable, but then fall to more desirable levels—may also be deemed as having symptoms related to hypertension. Patients with primary, essential, idiopathic hypertension, and secondary hypertension (e.g., renal hypertension and endocrine hypertension) are included in the category of individuals with hypertension.

The terms “diuretic” and “diuretic antihypertensive” are used herein to refer to drugs that affect sodium diuresis and volume depletion in a patient. Thus, diuretic antihypertensives include thiazides (such as hydrochlorothiazide, chlorothiazide, and chlorthalidone), metolazone, loop diuretics (such as furosemide, bumetanide, ethacrynic acid, piretanide and torsemide), and aldosterone antagonists (such as spironolactone, triamterene, and amiloride).

The terms “beta blocker” and “beta blocker antihypertensive” are used herein to refer to beta-adrenergic receptor blocking agents, e.g., drugs that block sympathetic effects on the heart and are generally most effective in reducing cardiac output and in lowering arterial pressure when there is increased cardiac sympathetic nerve activity. In addition, these drugs block the adrenergic nerve-mediated release of renin from the renal justaglomerular cells. Examples of this group of drugs include, but are not limited to, chemical agents such as propranolol, metoprolol, nadolol, atenolol, timolol, betaxolol, carteolol, pindolol, acebutolol, labetalol, and carvediol.

The terms “angiotensin converting enzyme inhibitor,” and “angiotensin converting enzyme inhibitor antihypertensive” are used herein to refer to drugs that are commonly known as ACE inhibitors. This group of drugs includes, for example, chemical agents such as captopril, benazepril, enalapril, enalaprilat, fosinopril, lisinopril, quinapril, ramipril, and trandolapril.

The term “sample” or “test sample” as used herein refers to a biological sample obtained for the purpose of diagnosis, prognosis, or evaluation. In certain embodiments, such a sample may be obtained for the purpose of determining the outcome of an ongoing condition or the effect of a treatment regimen on a condition. Preferred test samples include blood, serum, plasma, urine and saliva. In addition, one of skill in the art would realize that some samples would be more readily analyzed following a fractionation or purification procedure, for example, separation of whole blood into serum or plasma components.

The term “specific marker of hypertension treatment” as used herein refers to SNPs that are typically associated with genes in the Renin-angiotensin-aldosterone system, or other biochemical pathways, and which can be correlated with hypertension, but are not correlated with other types of disease. These systems, and others proposed to be involved in hypertension and affected by specific drugs, are in certain embodiments of the invention are candidates for gene/SNP sets to be used as system inputs for a predictive algorithm. These specific markers are described in detail hereinafter.

The term “non-specific marker of hypertension therapeutic action” as used herein refers to molecules that are typically general markers of cardiovascular disease. Such markers may be present in the event of myocardial injury, atherosclerotic plaque rupture, acute coronary syndrome, coagulation, and myocardial ischemia or necrosis but may also be present in general hypertensives.

Other non-specific markers of hypertension include genetic variants and protein products of genes encoding components in lipid metabolism such as CETP and LDLR.

The skilled artisan will recognize that nucleotide position can be found from reference sequence number (hereafter RS#) information by referring to a public database such as www.snpper.chip.orq. and that if no RS# exists one can refer to the literature for sequence information. An example of this latter case are mutations in the haptoglobin gene, which is referred by names of haptoglobin 1-1, haptoglobin 1-2, and haptoglobin 1-3. Detailed descriptions of the mutations and their respective genetic positions of these three are to be found by referring to Yano A. Yamamoto Y. Miyaishi S. Ishizu H. Haptoglobin genotyping by allele-specific polymerase chain reaction amplification. Acta Medica Okayama. 52(4):173-81, 1998 August. UI: 98454552, and Hill A V. Bowden D K. Flint J. Whitehouse D B. Hopkinson D A. Oppenheimer S J. Serjeantson S W. Clegg J B. A population genetic survey of the haptoglobin polymorphism in Melanesians by DNA analysis. American Journal of Human Genetics. 38(3):382-9, 1986 March., both incorporated in their entirety by reference.

The phrase “diagnosis” as used herein refers to methods by which the skilled artisan can estimate and even determine whether or not a patient is suffering from a given disease or condition. The skilled artisan often makes a diagnosis on the basis of one or more diagnostic indicators, i.e., a marker, the presence, absence, or amount of which is indicative of the presence, severity, or absence of the condition.

Similarly, a prognosis is often determined by examining one or more “prognostic indicators.” These are markers, the presence or amount of which in a patient (or a sample obtained from the patient) signal a probability that a given course or outcome, including treatment outcome, will occur. For example, when one or more prognostic indicators exhibit a certain pattern or level in samples obtained from such patients, the pattern or level may signal that the patient is at an increased probability for experiencing a future event in comparison to a similar patient exhibiting a different pattern or lower marker level. A certain pattern, level or a change in level of a prognostic indicator, which in turn is associated with an increased probability of disease recurrence or side effect such as obesity, is referred to as being “associated with an increased predisposition to an adverse outcome” in a patient. Preferred prognostic markers can predict the onset of delayed adverse events in a patient, or the chance of a person responding or not responding to a certain drug.

The term “correlating,” as used herein in reference to the use of diagnostic and prognostic indicators, refers to comparing the presence or amount of the indicator in a patient to its presence or amount in persons known to respond to a certain treatment; suffer from, or known to be at risk of, a given condition; or in persons known to be free of a given condition, i.e. “normal individuals”. For example, a SNP pattern or marker level in a patient sample can be compared to a SNP pattern or level known to be associated with response to a certain hypertension medication. The sample's marker pattern or level is said to have been correlated with a diagnosis; that is, the skilled artisan can use the marker pattern or level to determine whether the patient will respond to a certain medication, and prescribe accordingly. Alternatively, the sample's SNP pattern or marker level can be compared to a SNP pattern or marker level known to be associated with an adverse event (e.g., excessive dry cough or angiodemia), such as an SNP pattern or average level found in a population of normal individuals.

The skilled artisan will understand that, while in certain embodiments comparative measurements are made of the same diagnostic marker at multiple time points, one could also measure a given marker at one time point, and a second marker at a second time point, and a comparison of these markers may provide diagnostic information. The skilled artisan will also understand that proteomic or gene expression values may change in time, SNP patterns by definition are fixed in time.

The phrase “determining the prognosis” as used herein refers to methods by which the skilled artisan can predict the course or outcome of a condition in a patient. The term “prognosis” does not refer to the ability to predict the course or outcome of a condition with 100% accuracy, or even that a given course or outcome is predictably more or less likely to occur based on the presence, absence or levels of test markers. Instead, the skilled artisan will understand that the term “prognosis” refers to an increased probability that a certain course or outcome will occur; that is, that a course or outcome is more likely to occur in a patient exhibiting a given condition, such as nicotine dependence, when compared to those individuals not exhibiting the condition.

The skilled artisan will understand that associating a prognostic indicator with a predisposition to an adverse outcome is a statistical analysis. For example, a marker level of greater than 80 pg/mL may signal that a patient is more likely to suffer from an adverse outcome than patients with a level less than or equal to 80 pg/mL, as determined by a level of statistical significance. Additionally, a change in marker concentration from baseline levels may be reflective of patient prognosis, and the degree of change in marker level may be related to the severity of adverse events. Comparing two or more populations, and determining a confidence interval and/or a p value often determine statistical significance. See, e.g., Dowdy and Wearden, Statistics for Research, John Wiley & Sons, New York, 1983. Preferred confidence intervals of the invention are 90%, 95%, 97.5%, 98%, 99%, 99.5%, 99.9% and 99.99%, while preferred p values are 0.1, 0.05, 0.025, 0.02, 0.01, 0.005, 0.001, and 0.0001. Exemplary statistical tests and algorithmic methods for associating a prognostic indicator with a predisposition to an adverse outcome and success or failure on a treatment regime are described hereinafter.

Most existing statistical and computational methods for biomarker feature selection such as U.S. patent application 20040121343 and/or U.S. patent Ser. No. 10/225,082 have focused on differential expression of markers between diseased and control data sets. This metric is tested by simple calculation of fold changes, by t-test, and/or F test. These are based on variations of linear discriminant analysis (i.e., calculating some or the entire covariance matrix between features).

However, the majority of these data analysis methods are not effective for biomarker identification and disease diagnosis for the following reasons. First, although the calculation of fold changes or t-test and F-test can identify highly differentially expressed biomarkers, the classification accuracy of identified biomarkers by these methods, is, in general, not very high. This is because linear transforms typically extract information from only the second-order correlations in the data (the covariance matrix) and ignore higher-order correlations in the data. We have shown that proteomic datasets are inherently non-symmetric (unpublished data). For such cases, nonlinear transforms are necessary. Second, most scoring methods do not use classification accuracy to measure a biomarker's ability to discriminate between classes. Therefore, biomarkers that are ranked according to these scores may not achieve the highest classification accuracy among biomarkers in the experiments. Even if some scoring methods, which are based on classification methods, are able to identify biomarkers with high classification accuracy among all biomarkers in the experiments, the classification accuracy of a single marker cannot achieve the required accuracy in clinical diagnosis. Third, a simple combination of highly ranked markers according to their scores or discrimination ability is usually not be efficient for classification, as shown in the instant invention. If there is high mutual correlation between markers, then complexity increases without much gain.

Accordingly, the instant invention provides a methodology that can be used for biomarker feature selection and classification, and is applied in the instant application to prediction of response to various classes of hypertension medications.

How to Measure Various Markers

One of ordinary skill in the art know several methods and devices for the detection and analysis of the markers of the instant invention. With regard to polypeptides or proteins in patient test samples, immunoassay devices and methods are often used. These devices and methods can utilize labeled molecules in various sandwich, competitive, or non-competitive assay formats, to generate a signal that is related to the presence or amount of an analyte of interest. Additionally, certain methods and devices, such as biosensors and optical immunoassays, may be employed to determine the presence or amount of analytes without the need for a labeled molecule.

Preferably the markers are analyzed using an immunoassay, although other methods are well known to those skilled in the art (for example, the measurement of marker RNA levels). The presence or amount of a marker is generally determined using antibodies specific for each marker and detecting specific binding. Any suitable immunoassay may be utilized, for example, enzyme-linked immunoassays (ELISA), radioimmunoassay (RIAs), competitive binding assays, and the like. Specific immunological binding of the antibody to the marker can be detected directly or indirectly. Direct labels include fluorescent or luminescent tags, metals, dyes, radionuclides, and the like, attached to the antibody. Indirect labels include various enzymes well known in the art, such as alkaline phosphatase, horseradish peroxidase and the like. For an example of how this procedure is carried out on a machine, one can use the RAMP Biomedical device, called the Clinical Reader sup.™., which uses the fluoresent tag method, though the skilled artisan will know of many different machines and manual protocols to perform the same assay. Diluted whole blood is applied to the sample well. The red blood cells are retained in the sample pad, and the separated plasma migrates along the strip. Fluorescent dyed latex particles bind to the analyte and are immobilized at the detection zone. Additional particles are immobilized at the internal control zone. The fluorescence of the detection and internal control zones are measured on the RAMP Clinical Reader sup.™., and the ratio between these values is calculated. This ratio is used to determine the analyte concentration by interpolation from a lot-specific standard curve supplied by the manufacturer in each test kit for each assay.

The use of immobilized antibodies specific for the markers is also contemplated by the present invention and is well known by one of ordinary skill in the art. The antibodies could be immobilized onto a variety of solid supports, such as magnetic or chromatographic matrix particles, the surface of an assay place (such as microtiter wells), pieces of a solid substrate material (such as plastic, nylon, paper), and the like. An assay strip could be prepared by coating the antibody or a plurality of antibodies in an array on solid support. This strip could then be dipped into the test sample and then processed quickly through washes and detection steps to generate a measurable signal, such as a colored spot.

The analysis of a plurality of markers may be carried out separately or simultaneously with one test sample. Several markers may be combined into one test for efficient processing of a multiple of samples. In addition, one skilled in the art would recognize the value of testing multiple samples (for example, at successive time points) from the same individual. Such testing of serial samples will allow the identification of changes in marker levels over time. Increases or decreases in marker levels, as well as the absence of change in marker levels, would provide useful information about the disease status that includes, but is not limited to identifying the approximate time from onset of the event, the presence and amount of salvagable tissue, the appropriateness of drug therapies, the effectiveness of various therapies, identification of the severity of the event, identification of the disease severity, and identification of the patient's outcome, including risk of future events.

An assay consisting of a combination of the markers referenced in the instant invention may be constructed to provide relevant information related to differential diagnosis. Such a panel may be constucted using 1, 2, 3, 4, 5, 6, 7, 8, 9, 10, 15, 20, or more or individual markers. The analysis of a single marker or subsets of markers comprising a larger panel of markers could be carried out methods described within the instant invention to optimize clinical sensitivity or specificity in various clinical settings. The clinical sensitivity of an assay is defined as the percentage of those with the disease that the assay correctly predicts, and the specificity of an assay is defined as the percentage of those without the disease that the assay correctly predicts (Tietz Textbook of Clinical Chemistry, 2.sup.nd edition, Carl Burtis and Edward Ashwood eds., W. B. Saunders and Company, p. 496).

The analysis of markers could be carried out in a variety of physical formats as well. For example, the use of microtiter plates or automation could be used to facilitate the processing of large numbers of test samples. Alternatively, single sample formats could be developed to facilitate immediate treatment and diagnosis in a timely fashion, for example, in ambulatory transport or emergency room settings. Particularly useful physical formats comprise surfaces having a plurality of discrete, addressable locations for the detection of a plurality of different analytes. Such formats include protein microarrays, or “protein chips” (see, e.g., Ng and llag, J. Cell Mol. Med. 6: 329-340 (2002)) and capillary devices.

In another embodiment, the present invention provides a kit for the analysis of markers. Such a kit preferably comprises devises and reagents for the analysis of at least one test sample and instructions for performing the assay. Optionally the kits may contain one or more means for using information obtained from assays performed for a marker panel to rule in or out certain diagnoses.

Methodology of Marker Selection, Analysis, and Classification

Non-linear techniques for data analysis and information extraction are important for identifying complex interactions between markers that contribute to overall presentation of the clinical outcome. However, due to the many features involved in association studies such as the one proposed, the construction of these in-silico predictors is a complex process. Often one must consider more markers to test than samples, missing values, poor generalization of results, selection of free parameters in predictor models, confidence in finding a sub-optimal solution and others. Thus, the process for building a predictor is as important as designing the protocol for the association studies. Errors at each step can propagate downstream, affecting the generalizability of the final result.

We now provide an overview of our process of model development, describing the five main steps and some techniques that the instant invention will use to build an optimal biomarker panel of response for each clinical outcome. One of ordinary skill in the art will know that it is best to use a ‘toolbox’ approach to the various steps, trying several different algorithms at each step, and even combining several as in Step Five. Since one does not know a priori the distribution of the true solution space, trying several methods allows a thorough search of the solution space of the observed data in order to find the most optimal solutions (i.e. those best able to generalize to unseen data). One also can give more confidence to predictions if several independent techniques converge to a similar solution.

Data Pre-Processing

After assaying the patients for various markers, it is necessary to perform some basic data ‘inspection', such as identification of outliers, before starting a program of outcome prediction. Another task is performing data dimensional shifting in the case of discrete data sets such as SNP analysis. For instance, one can describe a three-state SNP vector either three-dimensionally (1,0,0);(0,1,0);(0,0,1) or two-dimensionally (0,0);(1,0);(0,0). For some algorithms, the latter description may have a direct effect on computational cost and classifier accuracy: one can, in effect, collapse several values to a single parameter. The advantage of single parameter is that one can reduce dimensionality with little or no effect on the selection of the optimal feature set. Following pre-processing, one can then perform univariate and multivariate statistical modeling to identify strongly correlative outcome variables and determine a baseline outcome analysis.

Missing Value Estimation

While the call rate and accuracy of high throughput methods are improving, genotype and proteomic data sets usually contain missing values. Missing values arise from missed genotype calls or from the combination of data collected under different protocols. If subsequent analysis requires complete data sets, repeating the experiment can be expensive and removing rows or columns containing missing values in the data set may be wasteful.

Missing values can be replaced with the most likely genotype based on frequency estimates for an individual marker. This row counting method may be sufficient when few markers are genotyped, but it is not optimal for genome wide scans since it does not consider correlation in the data. Other statistical approaches to estimating missing values apply genetic models of inheritance. In large-scale association studies of unrelated participants, lineage information is unavailable. For the dataset gathered in the instant invention, we will apply techniques that do not use complex models and take into account the possibly discrete nature of marker data when models are used. These methods fall into two categories: KNN-based and Bayesian-based methods.

KNN estimates the value of the missing data as the most prevalent genotype among the K Nearest Neighbors. For a data set consisting of M patients and N SNPs, the data is stored in an M by N matrix. For each row with a missing value in a single column, the algorithm locates the K nearest neighbors in the N-1 dimensional subspace. The K nearest neighbors then votes to replace the missing value under majority rule. Ties are broken by random draw. If there are n missing values present in a row, we find the nearest neighbors in the N-n subspace.

The only other consideration is what distance function to use to determine the K nearest neighbors. Typically, the Euclidean distance is well suited for continuous data and the Hamming distance for nominal data. The Hamming distance counts the number of different marker genotypes in the N-n subspace and does not impose an artificial ordinality as does the Euclidean distance. There are other options such as the Manhattan distance, the correlation coefficient, and others that may be used depending on the data set distribution.

In contrast, Bayesian imputation uses probabilities instead of distances to infer missing values. The objective is to draw an inference about a missing value for a matrix entry in the data set from the posterior probability of the missing value given the observed data, π(Yₘᵢₛₛ/Y_{obs}), where Y_{obs} is the set of N-n observed marker values and Yₘᵢₛₛ is the missing value. By Bayes's theorem, π(Yₘᵢₛₛ/Y_{obs}) can be expressed as follows:

$$π ( {Y}_{miss} \text{\vert} {Y}_{obs} ) = \frac{π ( {Y}_{obs} \text{\vert} {Y}_{mis} ) π ( {Y}_{miss} )}{{\sum }_{k = 1}^{m} π ( {Y}_{obs} \text{\vert} {Y}_{mis} ) π ( {Y}_{miss} )} \qquad ( 1 )$$

where π(Yₘᵢₛₛ) is the probability that a randomly selected missing entry will have the value Yₘᵢₛₛ, π(Y_{obs}|Yₘᵢₛₛ) is the probability of observing the N-n genotypes given Yₘᵢₛₛ, and the sum is over the m possible values for Yₘᵢₛₛ.

The likelihood model assumes that the probabilities π(Y_{obs}|Yₘᵢₛₛ) can be expressed as functions of unknown parameters of the genotypes Yₘᵢₛₛ:

$$π ( {Y}_{obs = g} \text{\vert} {Y}_{miss = k} ) = π ( {y}_{g 1} \text{\vert} {θ}_{1 k} ) π ( {y}_{g 2} \text{\vert} {θ}_{2 k} ) … π ( {y}_{gn} \text{\vert} {θ}_{nk} ) = {\prod }_{i = 1}^{N - n} π ( {y}_{gi} \text{\vert} {θ}_{ik} ) \qquad ( 2 )$$

where θᵢₖ are unknown parameters of Yₘᵢₛₛ for the N-n observed markers, y_{gi} is the i th marker in the set of Y_{obs} markers, and θ(y_{gi}|θᵢₖ) is the probability of observing y_{gi} given the parameter θᵢₖ of the marker value Yₘᵢₛₛ for variable i. The model is based on the assumption that the probability of observing y_{gi} is independent of the probability of observing y_{gi} for each marker value Yₘᵢₛₛ with i≠j.

Missing values are imputed as follows. For each marker for which there is a missing value, the probabilities θ(y_{gi}|θᵢₖ) are estimated based on the observed markers. Using Bayes' theorem, the posterior probability θ(Yₘᵢₛₛ|Y_{obs}) is calculated. We then sample Yₘᵢₛₛ from the posterior. This approach treats the missing value problem as a supervised learning problem in which posterior probability is learned from the pattern of observed markers.

Feature Selection

Following missing value replacement, the third step in the predictive panel building process is to perform feature selection on the dataset; this is perhaps the most important step in the predictor development process. Feature selection serves two purposes: (1) to reduce dimensionality of the data and improve classification accuracy, and (2) to identify biomarkers that are relevant to the cause and consequences of disease and drug response.

A feature selection algorithm (FSA) is a computational solution that given a set of candidate features selects a subset of relevant features with the best commitment among its size and the value of its evaluation measure. However, the relevance of a feature, as seen from the classification perspective, may have several definitions depending on the objective desired. An irrelevant feature is not useful for classification, but not all relevant features are necessarily useful for classification.

Another problem from which many classification methods suffer is the curse of dimensionality. That is, as the number of features in a classification task increases, the time requirements for an algorithm grow dramatically, sometimes exponentially. Therefore, when the set of features in the data is sufficiently large, many classification algorithms are simply intractable. This problem is further exacerbated by the fact that many features in a learning task may either be irrelevant or redundant to other features with respect to predicting the class of an instance. In this context, such features serve no purpose except to increase classification time.

FSAs can be divided into two categories based on whether or not feature selection is done independently of the learning algorithm used to construct the classifier. If the feature selection is independent of the learning algorithm, the technique is said to follow a filter approach. Otherwise, it is said to follow a wrapper approach. While the filter approach is generally computationally more efficient than the wrapper approach, a drawback is that an optimal selection of features may not be independent of the inductive and representational biases of the learning algorithm to be used to construct the classifier.

SFS/SBS

A sequential forward search (SFS), or backward (SBS), is a process that uses an iterative technique for feature selection. In this wrapper technique, one feature at a time is added (SFS) or deleted (SBS) to a set of pre-selected features, and iterated according to a performance metric until the ‘optimal’ set of features are obtained. For example, SFS is a technique that starts with all possible two-variable input combinations from the entire data set and then builds, one variable at a time, until an optimally performing combination of variables is identified. For instance, with 9 input variables labeled 1-9 (each with a binary descriptor), the two-variable combinations would comprise 1|2, 1|3, 1|4, 1|5, 1|6, 1|7, 1|8, 1|9, 2|3, 2|4, 2|5, 2|6 . . . 8|9. These input combinations are each used in training a classifier using the collected data. The combinations that perform the best (evaluated using leave-one-out cross validation; top 10%, for example) are selected for continued addition of variables. Let us say that 2|3 is selected as one of the top performers, it would then be coupled to each of the other variables, not including those variables that are already included in the combination. This would result in 2|3|1, 2|3|4, 2|3|5, 2|3|6, 2|3|7, 2|3|8 and 2|3|9. This coupling is performed for all of the top two-variable performers. The resultant three-variable input combinations are used to train a classifier using the collected data and then evaluated. The top performers are selected and then coupled again with all variables in the group, again used to train a classifier. This is repeated until a maximal predictive accuracy is achieved. In our experience we have noticed a well defined ‘hump’ at the point where the addition of variables into the system results begins to contribute to degradation of system performance.

SBS starts with the full set of features and eliminates those based upon a performance metric. Although in theory, going backward from the full set of features may capture interacting features more easily, the drawback of this method is that it is computationally expensive.

An example of this is described in U.S. patent application Ser. No. 09/611,220, incorporated in entirety with all figures by reference, which uses a variation on the SBS technique. In this method, a Genetic Algorithm (please see section on classifiers) is used in combination with a neural network to create and select child features based upon a fitness ranking that takes into effect multiple performance measures such as sensitivity and specificity. Only top-ranked child features are used in iterating the algorithm forward.

SFFS

The SFS algorithm suffers from a so-called nesting effect. That is, once a feature has been chosen, there is no way for it to be discarded. To overcome this problem, the sequential forward floating algorithm (SFFS) was proposed. SFFS is an exponential cost algorithm that operates in a sequential manner. In each selection step SFFS performs a forward step followed by a variable number of backward ones. In essence, a feature is first unconditionally added and then features are removed as long as the generated subsets are the best among their respective size. The algorithm is so-called because it has the characteristic of floating around a potentially good solution of the specified size.

E-RFE

The Recursive Feature Elimination (RFE) is a well-known feature selection method for support vector machines (SVMs, please see section on classifiers). As a brief overview, a SVM realizes a classification function

$$f ( x ) = {\sum }_{i = 1}^{N} {α}_{i} {γ}_{i} K ( {x}_{i} , x ) + b ,$$

where the coefficients α=(αᵢ) and b are obtained by training over a set of examples S={(xᵢ, yᵢ}I=1, . . . , N, xᵢεRⁿ, yᵢε{−1, 1} and) K(xᵢx) is the chosen kernel. In the linear case, the SVM expansion defines the hyperplane

$$f ( x ) = \text{<} w , x \text{>} + b , \\ with$$

$$w = {\sum }_{i = 1}^{N} {α}_{i} {γ}_{i} {x}_{i} .$$

The idea is to define the importance of a feature for a SVM in terms of its contribution to a cost function J(α). At each step of the RFE procedure, a SVM is trained on the given data set, J is computed and the feature less contributing to J is discarded. In the case of linear SVM, the variation due to the elimination of the i-th feature is δJ(i)=wᵢ²; in the non linear case, δJ(i)=1/2α^{t}Zα−1/2α^{t}Z(−i) α, where Zᵢⱼ=yᵢyⱼK (xᵢ, xⱼ). The heavy computational cost of RFE is a function of the number of variables, as another SVM must be trained each time a variable is removed. In the standard RFE algorithm we would eliminate just one of the many features corresponding to a minimum weight, while it would be convenient to remove all of them at once. We will go further in the instant invention by developing an ad hoc strategy for an elimination process based on the structure of the weight distribution. This strategy was first described by Furlanello (24). We introduce an entropy function H as a measure of the weight distribution. To compute the entropy, we split the range of the weights, normalized in the unit interval, into nᵢₙₜ intervals (with nᵢₙₜ=\sqrt{#R}), and we compute for each interval the relative frequencies

$${p}_{i} = \frac{\# δ J ( i )}{\# R} , \\ i = 1 , … , {n}_{int}$$

Entropy is then defined as the following function:

$$H = - {\sum }_{i = 1}^{{n}_{int}} {p}_{i} {log}_{2} {p}_{i}$$

The following inequality immediately descends from the definition of entropy: 0≦H≦log₂nᵢₙₜ, the two bounds corresponding to the situations:
H=0; or all the weights lie in one interval;
H=log₂nᵢₙₜ; or all the intervals contain the same number of weights.
   The new entropy-based RFE (E-RFE) algorithm eliminates chunks of features at every loop, with two different procedures applied for lower or higher values of H. The distinction is needed to remove many features that have a similar (low) weight while preserving the residual distribution structure, and also allowing for differences between classification problems. E-RFE has been shown to speed up RFE by a factor of 100.

URG

One filter method especially suited for ordinal data has been developed recently by the authors of the instant invention, and offers clearly interpretable results on such data. The feature selection aspect, tentatively named URG, or Universal Regressor Gauge, is a general method for scoring and ranking the predictive sensitivity of input variables by fitting the gauge, or the scaling, on each of the input variables subject to both predictive accuracy of a nonparametric regression, and a penalty on the L1 norm of the vector of scaling parameters. The result is a sampled-gradient local minimum solution that does not require assumptions of linearity or exhaustive power-set sampling of subsets of variables. The approach penalizes the gauge θ, or the set of scaling parameters (θ₁, θ₂, . . . , θₙ), applied to each of the input variables. The authors of the instant invention generalized this method to potentially nonlinear, nonparametric models of arbitrary complexity using a kernel-based nonparametric regressor. The penalty on the gauge is regularized by a coefficient λ that is scanned across a range of values to put progressively more downward pressure on the scaling parameters, forcing the scale (and the resulting significance in distance-based regression) downward first on those variables that can be most easily eliminated without sacrificing accuracy. Because this process is analog in the state-space of the gauge, nonlinear interactions between subsets can be investigated in a continuous manner, even if the variables themselves are discrete-valued.

Other FSAs complentated, but not limited to, to be used in the instant invention include HITON Markov Blankets and Bayesian filters.

Classification

The fourth step in the predictor-building process is classification. In the supervised learning task, one is given a training set of labeled fixed-length feature vectors, from which to induce a classification model. This model, in turn, is used to predict the class label for a set of previously unseen instances. Thus, in building a classification model, the information about the class that is inherent in the features is of utmost importance. The dataset that the classifier is trained upon is broken up generally into three different sets: Training, Testing, and Evaluation. This is required since when using any classifier, the use of distinct subsets of the available data for training and testing is required to ensure generalizability. The parameters of the classifier are set with respect to the training data set, and judged versus competitors on the testing data set, and validated on the evaluation data set. To avoid over-training (i.e., memorization of features in a specific data set that are not applicable in a general manner) this succession of training steps is discontinued when the error on the validation set begins to increase significantly. We use the error on the evaluation data set as an estimate of how well we can expect our classifier to perform on new testing data as it becomes available. This estimate can be measured by 10× leave-one-out-cross-validation on the evaluation set (100× in cases of low sample number), or batch evaluation on larger data sets.

Classifiers complimentated for the instant invention include, but are not limited to, neural networks, support vector machines, genetic algorithms, kernel-based methods, and tree-based methods.

Neural Networks

One tool to use construct classifiers is that of a mapping neural network. The flexibility of neural nets to generically model data is derived through a technique of “learning”. Given a list of examples of correct input/output pairs, a neural net is trained by systematically varying its free parameters (weights) to minimize its chi-squared error in modeling the training data set. Once these optimal weights have been determined, the trained net can be used as a model of the training data set. If inputs from the training data are fed to the neural net, the net output will be roughly the correct output contained in the training data. The nonlinear interpolatory ability manifests itself when one feeds the net sets of inputs for which no examples appeared in the training data. A neural net “learns” enough features of the training data set to completely reproduce it (up to a variance inherent to the training data); the trained form of the net acts as a black box that produces outputs based on the training data.

Neural networks typically have a number of ad hoc parameters, such as selection of the number of hidden layers, the number of hidden-layer neurons, parameters associated with the learning or optimization technique used, and in many cases they require a validation set for a stopping criterion. In addition, neural network weights are trained iteratively, producing problems with convergence to local minima. We have developed several types of neural networks that solve these problems. Our solutions involve nonlinearly transforming the input pattern fed into the neural network. This transformation is equivalent to feature selection (though one still needs as many inputs into the classifier) and can be quite powerful when combined with the independent feature selection techniques previously described.

Genetic Algorithms

Genetic algorithms (GAs) typically maintain a constant sized population of individual solutions that represent samples of the space to be searched. Each individual is evaluated on the basis of its overall “fitness” with respect to the given application domain. New individuals (samples of the search space) are produced by selecting high performing individuals to produce “offspring” that retain features of their “parents”. This eventually leads to a population that has improved fitness with respect to the given goal.

New individuals (offspring) for the next generation are formed by using two main genetic operators: crossover and mutation. Crossover operates by randomly selecting a point in the two selected parents gene structures and exchanging the remaining segments of the parents to create new offspring. Therefore, crossover combines the features of two individuals to create two similar offspring. Mutation operates by randomly changing one or more components of a selected individual. It acts as a population perturbation operator and is a means for inserting new information into the population. This operator prevents any stagnation that might occur during the search process.

GAs have demonstrated substantial improvement over a variety of random and local search methods. This is accomplished by their ability to exploit accumulating information about an initially unknown search space in order to bias subsequent search into promising subspaces. Since GAs are basically a domain independent search technique, they are ideal for applications where domain knowledge and theory is difficult or impossible to provide.

SVMs

The key idea behind support vector machines (SVMs, Vapnik, 1995) is to map input vectors (i.e., patient-specific data) into a high dimensional space, and to construct in that space hyperplanes with a large margin. These hyperplanes can be thought of as boundaries separating the categories of the dataset, in this case response and non-response. The support vector machine solution proposes to find the hyperplane separating the classes. This plane is determined by the parameters of a decision function, which is used for classification. The SVM is based on the fact that there is a unique separating hyperplane that maximizes the margin between the classes.

The task of finding the hyperplane is reduced to minimizing the Lagrangian, a function of the margin and constraints associated with each input vector. The constraints depend only on the dot product of an input element and the solution vector. In order to minimize the Langrangian, the Lagrange multipliers must either satisfy those constraints or be exactly zero. Elements of the training set for which the constraints are satisfied are the so-called support vectors. The support vectors parameterize the decision function and lie on the boundaries of the margin separating the classes.

In many cases, SVMs are typically more accurate, give greater data understanding, and are more robust than other machine learning methods. Data understanding comes about because SVMs extract support vectors, which as described above are the borderline cases. Exhibiting such borderline cases allow us to identify outliers, to perform data cleaning, and to detect confounding factors. In addition, the margins of the training examples (how far they are from the decision boundary) provide useful information about the relevance of input variables, and allow the selection of the most predictive variable. SVMs are often successful even with sparse data (few examples), biased data (more examples of one category), redundant data (many similar examples), and heterogeneous data (examples coming from different sources). However, they are known to work poorly on discrete data.

In another preferred embodiment of the present invention, regression techniques are used to deliver a diagnostic or prognostic prediction using the markers declared previously. These are well-known by those of ordinary skill in the art, however a short discussion follows. For more detail, one is referred to Kleinbaum et al., Applied Regression Analysis and Multivariable Methods, Third Edition, Duxbury Press, 1998.

In the discussion of weighted least squares a need was found for a method to fit Y to more than one X. Further, it is common that the response variable Y is related to more than one regressor variable simultaneously. If a valid description of the relationship between Y and any of these response variables is to be obtained, all must be considered. Also, exclusion of any important regressor variables will adversely affect predictions of Y. In general, the equation to be considered becomes
Y=b0+b1X1+b2X2+ . . . +bKXK
The Xs may be any relevant regressor variables. Often one X is a (nonlinear) transformation of another. For example, X2=ln(X1).

When dealing with multiple linear regression, fits to data are no longer lines. For example, with K=2, the resulting fit would describe a plane in three dimensional space with “slopes” bhat 1 and bhat 2 intersecting the Y axis at bhat 0. Beyond K=2 the resulting fit becomes difficult to visualize. The terminology regression surface is often used to describe a multiple linear regression fit.

Assumptions required for application of least squares methodology to multiple linear regression equations are similar to those cited for the simple linear case. For example, the true relationship between Y and the various Xs must be as given by the linear equation and the spread of the errors must be constant across values of all Xs. Also, a limit exists to the number of Xs that can be considered. Specifically, K+1 must be less than or equal to the sample size n for a unique set of bhats to be found.

In theory, least squares estimates of b 0, . . . , b K are found just as in the simple linear case. The estimates bhat 0, . . . , bhat K are the solution from minimizing sum (Yi−b0−b1X1i−, . . . −bkXki)sup2.

The description of the resulting equations and associated summary statistics is best made using matrix algebra. The computations are best carried out using a computer.

The relationship between Y and X or Y and several Xs is not always linear in form despite transformations that can be applied to resulted in a linear relationship. In some instances such a transformation may not exist and in others theoretical concerns may require analysis to be carried out with the untransformed equation.

Least squares methodology can be used to solve nonlinear regression problems. For the above equation the least squares estimates of the parameters would be the solution of the minimization of sum(W−A (1−e sup Bt )sup C)sup 2

Application of calculus leads to three equations whose solution requires an iterative technique. For all but the simplest of cases, solving nonlinear least squares problems involves use of computer-based algorithms. A multitude of such algorithms exist emphasizing the number of problems whose valid solution requires the nonlinear least squares technique.

Several variations of nonlinear regression exist, which one of ordinary skill in the art will be aware. One preferred case in the present invention is the use of deterministic greedy algorithms for building sparse nonlinear regression models from observational data. In this embodiment, the objective is to develop efficient numerical schemes for reducing the training and runtime complexities of nonlinear regression techniques applied to massive datasets. In the spirit of Natarajan's greedy algorithm (Natarajan, 1995), the procedure is to iteratively minimize a loss function subject to a specified constraint on the degree of sparsity required of the final model or an upper bound on the empirical error. There exist various greedy criteria for basis selection and numerical schemes for improving the robustness and computational efficiency of these algorithms.

In another preferred embodiment of the present invention, a kernel-based method is trained to deliver a diagnostic or prognostic prediction using the markers declared previously. One such method is Kernel Fisher's Discriminant (KFD). Fisher's discriminant (Fisher, 1936) is a technique to find linear functions that are able to discriminate between two or more classes. Fisher's idea was to look for a direction w that separates the class means values well (when projected onto the found direction) while achieving a small variance around these means. The hope is that it is easy to differentiate between either of the two classes from this projection with a small error. The quantity measuring the difference between the means is called between class variance and the quantity measuring the variance around these class means is called within class variance, respectively. The goal is to find a direction that maximizes the between class variance while minimizing the within class variance at the same time. As this technique has been around for almost 70 years it is well known and widely used to build classifiers.

Unfortunately, as previously discussed, many biological datasets are not solvable using linear techniques. Therefore, one of the classifiers we use is a non-linear variant of Fisher's discriminant. This non-linearization is made possible through the use of kernel functions, a “trick” that is borrowed from support vector machines (Boser et al., 1992). Kernel functions represent a very principled and elegant way of formulating non-linear algorithms, and the findings that are derived from using them have clear and intuitive interpretations.

In the KFD technique (Mika, 1999), one first maps the data into some feature space F through some non-linear mapping Φ. One then computes Fisher's linear discriminant in this feature space, thus implicitly yielding a non-linear discriminant in input space. In a methodology similar to SVMs, this mapping is defined in terms of a kernel function k(x,y)=(Φ(x)·Φ(y)). The training examples (i.e. the data vector containing all marker values for each patient) can in turn be expanded in terms of this kernel function as well. From this relationship one can write a formulation of the between and within class variance in terms of dot products of the kernel function and training patterns and thus find Fisher's linear discriminant in F by maximizing the ratio of these two quantities.

In another preferred embodiment of the present invention, an algorithm using Bayesian learning is trained to deliver a diagnostic or prognostic prediction using the markers declared previously. See Pearl, J. (1988). Probabilistic Reasoning in Intelligent Systems: networks of plausible inference, Morgan Kaufmann, for an overview of Bayesian learning.

While Bayesian networks (BNs) are powerful tools for knowledge representation and inference under conditions of uncertainty, they were not considered as classifiers until the discovery that Naïve-Bayes, a very simple kind of BNs that assumes the attributes are independent given the class node, are surprisingly effective. See Langley, P., Iba, W. and Thompson, K. (1992). An analysis of Bayesian classifiers. In Proceedings of AAAI-92 pp. 223-228.

A Bayesian network B is a directed acyclic graph (DAG), where each node N represents a domain variable (i.e., a dataset attribute), and each arc between nodes represents a probabilistic dependency, quantified using a conditional probability distribution (CP table) for each node n.sub.i. A BN can be used to compute the conditional probability of one node, given values assigned to the other nodes; hence, a BN can be used as a classifier that gives the posterior probability distribution of the class node given the values of other attributes. A major advantage of BNs over many other types of predictive models, such as neural networks, is that the Bayesian network structure represents the inter-relationships among the dataset attributes. One of ordinary skill in the art can easily understand the network structures and if necessary modify them to obtain better predictive models. By adding decision nodes and utility nodes, BN models can also be extended to decision networks for decision analysis. See Neapolitan, R.E. (1990), Probabilistic reasoning in expert systems: theory and algorithms, Cohn Wiley& Sons.

Applying Bayesian network techniques to classification involves two sub-tasks: BN learning (training) to get a model and BN inference to classify instances. Learning BN models can be very efficient. As for Bayesian network inference, although it is NP-hard in general (See for instance Cooper, G. F. (1990) Computational complexity of probabilistic inference using Bayesian belief networks, In Artificial Intelligence, 42 (pp. 393-405).), it reduces to simple multiplication in a classification context, when all the values of the dataset attributes are known.

The two major tasks in learning a BN are: learning the graphical structure, and then learning the parameters (CP table entries) for that structure. One skilled in the art knows it is easy to learn the parameters for a given structure that are optimal for a given corpus of complete data, the only step being to use the empirical conditional frequencies from the data.

There are two ways to view a BN, each suggesting a particular approach to learning. First, a BN is a structure that encodes the joint distribution of the attributes. This suggests that the best BN is the one that best fits the data, and leads to the scoring based learning algorithms, that seek a structure that maximizes the Bayesian, MDL or Kullback-Leibler (KL) entropy scoring function. See for instance Cooper, G. F. and Herskovits, E. (1992). A Bayesian Method for the induction of probabilistic networks from data. Machine Learning, 9 (pp. 309-347). Second, the BN structure encodes a group of conditional independence relationships among the nodes, according to the concept of d-separation. See for instance Pearl, J. (1988). Probabilistic Reasoning in Intelligent Systems: networks of plausible inference, Morgan Kaufmann. This suggests learning the BN structure by identifying the conditional independence relationships among the nodes. These algorithms are referred as CI-based algorithms or constraint-based algorithms. See for instance Cheng, J., Bell, D. A. and Liu, W. (1997a). An algorithm for Bayesian belief network construction from data. In Proceedings of Al&STAT'97 (pp. 83-90), Florida.

Friedman et al. (1997) show theoretically that the general scoring-based methods may result in poor classifiers since a good classifier maximizes a different function —viz., classification accuracy. Greiner et al. (1997) reach the same conclusion, albeit via a different analysis. Moreover, the scoring-based methods are often less efficient in practice. The preferred embodiment is Cl-based learning algorithms to effectively learn BN classifiers.

The present invention envisions using, but is not limited to, the following five classes of BN classifiers: Naïve-Bayes, Tree augmented Naïve-Bayes (TANs), Bayesian network augmented Naïve-Bayes (BANs), Bayesian multi-nets and general Bayesian networks (GBNs). By use of this methodology it is possible to build a predictive model of the data.

These models can be put on firm theoretical foundations of statistics and probability theory, i.e. in a Bayesian setting. The computation required for inference in these models include optimization or marginalisation over all free parameters in order to make predictions and evaluations of the model. Inference in all but the very simplest models is not analytically tractable, so approximate techniques such as variational approximations and Markov Chain Monte Carlo may be needed. Models include probabilistic kernel based models, such as Gaussian Processes and mixture models based on the Dirichlet Process.

Ensemble Networks

The final step in predictor development, assembly of committee, or ensemble, networks. It is common practice to train many different candidate networks and then to select the best, on the basis of performance on an independent validation set, for instance, and to keep this network, discarding the rest. There are two disadvantages to this approach. First, the effort involved in training the remaining networks is wasted. Second, the generalization performance on the validation set has a random component due to noise on the data, and so the network that had the best performance on the validation set might not be the one with the best performance on the new test set.

These drawbacks can be overcome by combining the networks together to form a committee. This can lead to significant improvements in the predictions on new data while involving little additional computational effort. In fact, the performance of a committee can be better than the performance of the best single network in isolation. The error due to the committee can be shown to be:
E_{COM}=1/L E_{AV}
Where L is the number of committee members and EAV the average error contributed to the prediction by a single member of the committee. Typically, some useful reduction in error is obtained, and the method is trivial to implement.

The challenging problem of integration is to decide which one(s) of the classifiers to rely on or how to combine the results produced by the base classifiers. One of the most popular and simplest techniques used is called majority voting. In the voting technique, each base classifier is considered as an equally weighted vote for that particular prediction. The classification that receives the largest number of votes is selected as the final classification (ties are solved arbitrarily). Often, weighted voting is used: each vote receives a weight, which is usually proportional to the estimated generalization performance of the corresponding classifier. Weighted Voting (WV) works usually much better than simple majority voting.

Boosting Networks

Boosting has been found to be a powerful classification technique with remarkable success on a wide variety of problems, especially in higher dimensions. It aims at producing an accurate combined classifier from a sequence of weak (or base) classifiers, which are fitted to iteratively reweighted versions of the data.

In each boosting iteration, m, the observations that have been misclassified at the previous step have their weights increased, whereas the weights are decreased for those that were classified correctly. The m^{th} weak classifier f(m) is thus forced to focus more on individuals that have been difficult to classify correctly at earlier iterations. In other words, the data is re-sampled adaptively so that the weights in the re-sampling are increased for those cases most often misclassified. The combined classifier is equivalent to a weighted majority vote of the weak classifiers.

Entropy-Based

One efficient way to construct an ensemble of diverse classifiers is to use different feature subsets. To be effective, an ensemble should consist of high-accuracy classifiers that disagree on their predictions. To measure the disagreement of a base classifier and the whole ensemble, we calculate the diversity of the base classifier over the instances of the validation set as an average difference in classifications of all possible pairs of classifiers including the given one. A measure of this is based on the concept of entropy:

$$div\_ent = \frac{1}{N} {\sum }_{i = 1}^{N} {\sum }_{k = 1}^{l} - \frac{{N}_{k}^{l}}{S} \cdot log ( \frac{{N}_{k}^{l}}{S} )$$

where N is the number of instances in the data set, S is the number of base classifiers, I is the number of classes, and Nₖ^{l} is the number of base classifiers that assign instance i to class k.

### BRIEF DESCRIPTION OF THE DRAWINGS

In the following, the invention will be explained in further detail with reference to the drawings, in which:

FIG. 1 is a chart illustrating ROC curves for the 5× cross-validation data and the naïve data set results using the primary model developed for prediction of beta-blocker response;

FIG. 2 is a chart showing the univariate statistically significance for each of the SNPs in the beta-blocker model with respect to response/non-response outcome;

FIG. 3 is a chart illustrating ROC curves for the 5× cross-validation data and the naïve data set results using the primary model developed for prediction of ACE Inhibitor response;

FIG. 4 is a chart showing the univariate statistically significance for each of the SNPs in the beta-blocker model with respect to response/non-response outcome;

FIG. 5 is a table with details on SNPs genotyped.for elucidation of hypertension response; and

FIG. 6 is a table of correlative values of various SNPs with hypertension response.

### DETAILED DESCRIPTION OF THE INVENTION

In accordance with the present invention, there are provided methods and apparatus for the identification and use of a panel of markers for the diagnosis of cardiovascular illness, particularity stroke and its sub-types.

Method for Defining Panels of Markers

In practice, data may be obtained from a group of subjects. The subjects may be patients who have been tested for the presence or level of certain markers. Such markers and methods of patient extraction are well known to those skilled in the art. A particular set of markers may be relevant to a particular condition or disease. The method is not dependent on the actual markers. The markers discussed in this document are included only for illustration and are not intended to limit the scope of the invention. Examples of such markers and panels of markers are described in the instant invention and the incorporated references.

Well-known to one of ordinary skill in the art is the collection of patient samples. A preferred embodiment of the instant invention is that the samples come from two or more different sets of patients, one a disease group of interest and the other(s) a control group, which may be healthy or diseased in a different indication than the disease group of interest. For instance, one might want to look at the difference in blood-borne markers between patients who have had stroke and those who had stroke mimic to differentiate between the two populations.

The blood samples are assayed, and the resulting set of values are put into a database, along with outcome, also called phenotype, information detailing the illness type, for instance stroke mimic, once this is known. Additional clinical details such as time from onset of symptoms and patient physiological, medical, and demographics, the sum total called patient characteristics, are put into the database. The time from onset is important to know as initial marker values from onset of symptoms can change significantly over time on a timeframe of tens of minutes. Thus, a marker may be significant at one point in the patient history and not at another in predicting diagnosis or prognosis of cardiovascular disease, damage or injury. The database can be simple as a spreadsheet, i.e. a two-dimensional table of values, with rows being patients and columns being filled with patient marker and other characteristic values.

From this database, a computerized algorithm can first perform pre-processing of the data values. This involves normalization of the values across the dataset and/or transformation into a different representation for further processing. The dataset is then analyzed for missing values. Missing values are either replaced using an inputation algorithm, in a preferred embodiment using KNN or MVC algorithms, or the patient attached to the missing value is exised from the database. If greater than 50% of the other patients have the same missing value then value can be ignored.

Once all missing values have been accounted for, the dataset is split up into three parts: a training set comprising 33-80% of the patients and their associated values, a testing set comprising 10-50% of the patients and their associated values, and a validation set comprising 1-50% of the patients and their associated values. These datasets can be further sub-divided or combined according to algorithmic accuracy. A feature selection algorithm is applied to the training dataset. This feature selection algorithm selects the most relevant marker values and/or patient characteristics. Preferred feature selection algorithms include, but are not limited to, Forward or Backward Floating, SVMs, Markov Blankets, Tree Based Methods with node discarding, Genetic Algorithms, Regression-based methods, kernel-based methods, and filter-based methods.

Feature selection is done in a cross-validated fashion, preferably in a naïve or k-fold fashion, as to not induce bias in the results and is tested with the testing dataset. Cross-validation is one of several approaches to estimating how well the features selected from some training data is going to perform on future as-yet-unseen data and is well-known to the skilled artisan. Cross validation is a model evaluation method that is better than residuals. The problem with residual evaluations is that they do not give an indication of how well the learner will do when it is asked to make new predictions for data it has not already seen. One way to overcome this problem is to not use the entire data set when training a learner. Some of the data is removed before training begins. Then when training is done, the data that was removed can be used to test the performance of the learned model on “new” data.

Once the algorithm has returned a list of selected markers, one can optimize these selected markers by applying a classifer to the training dataset to predict clinical outcome. A cost function that the classifier optimizes is specified according to outcome desired, for instance an area under receiver-operator curve maximizing the product of sensitivity and specificity of the selected markers, or positive or negative predictive accuracy. Testing of the classifier is done on the testing dataset in a cross-validated fashion, preferably naïve or k-fold cross-validation. Further detail is given in U.S. patent application Ser. No. 09/611,220, incorporated by reference. Classifiers map input variables, in this case patient marker values, to outcomes of interest, for instance, prediction of stroke sub-type. Preferred classifiers include, but are not limited to, neural networks, Decision Trees, genetic algorithms, SVMs, Regression Trees, Cascade Correlation, Group Method Data Handling (GMDH), Multivariate Adaptive Regression Splines (MARS), Multilinear Interpolation, Radial Basis Functions, Robust Regression, Cascade Correlation+Projection Pursuit, linear regression, Non-linear regression, Polynomial Regression, Regression Trees, Multilinear Interpolation, MARS, Bayes classifiers and networks, and Markov Models, and Kernel Methods.

The classification model is then optimized by for instance combining the model with other models in an ensemble fashion. Preferred methods for classifier optimization include, but are not limited to, boosting, bagging, entropy-based, and voting networks. This classifier is now known as the final predictive model. The predictive model is tested on the validation data set, not used in either feature selection or classification, to obtain an estimate of performance in a similar population.

The predictive model can be translated into a decision tree format for subdividing the patient population and making the decision output of the model easy to understand for the clinician. The marker input values might include a time since symptom onset value and/or a threshold value. Using these marker inputs, the predictive model delivers diagnositic or prognostic output value along with associated error. The instant invention anticipates a kit comprised of reagents, devices and instructions for performing the assays, and a computer software program comprised of the predictive model that interprets the assay values when entered into the predictive model run on a computer. The predictive model receives the marker values via the computer that it resides upon.

Once patients are exhibiting symptoms of cardiovascular illness, for instance hypertension, a blood sample is drawn from the patient using standard techniques well known to those of ordinary skill in the art and assayed for various blood-borne markers of cardiovascular illness. Assays can be preformed through immunoassays or through any of the other techniques well known to the skilled artisan. In a preferred embodiment, the assay is in a format that permits multiple markers to be tested from one sample, such as the Luminex platform.TM. and/or in a rapid fashion, defined to be under 30 minutes and in the most preferred enablement of the instant invention, under 15 minutes. The values of the markers in the samples are inputed into the trained, tested, and validated algorithm residing on a computer, which outputs to the user on a display and/or in printed format on paper and/or transmits the information to another display source the result of the algorithmn calculations in numerical form, a probability estimate of the clinical diagnosis of the patient. There is an error given to the probability estimate, in a preferred embodiment this error level is a confidence level. The medical worker can then use this diagnosis to help guide treatment of the patient.

### EXAMPLE I

A study of 1108 hypertensive patents taking ACE Inhibitors, Calcium Channel Blockers, and Beta Blockers to control their blood pressure was performed. Normal blood pressure for an adult is around 120/80 mmHg. We defined a hypertensive patient as a patient with BP above 149/90 mmHg. The patients were newly diagnosed and not on any previous hypertensive medication. Drugs in each class were not distinguished between, and we also did not distinguish non-response due to lack of BP reduction vs. non-response due to adverse effects.

The definition of a “Responder” used was a patient who is hypertensive, takes an anti-hypertensive medication, and has consistent normotensive BP after medication treatment without adverse side effects.

We used the initial diagnosis of hypertension by the physician on the patient's chart as sufficient information to classify the patient as hypertensive. A BP at diagnosis, or a series of prior hypertensive BP measurements reinforces this diagnosis.

We set a minimum duration of 6 months since initial diagnosis of hypertension. We further reinforce this by a minimum 6 months duration of one medication therapy if a patient has switched medication.

To be classified as normotensive after medication, patients must have at least 3 normotensive BP measurements over a minimum 6-month duration of therapy, and no more than 1 hypertensive measurement after medication start date. Patients must also have no adverse side effects noted on charts during the duration of medication. If a patient was not quite normotensive, yet, doctor's discretion kept the patient on the medication for over a year, we also allowed responder classification as a 40+mmHg reduction in systolic or diastolic blood pressure.

The simplest definition of a “Non-responder” is a patient who is hypertensive, takes an anti-hypertensive medication, and has continues to have a hypertensive BP after medication treatment, experiences adverse side effects, or must revert to alternative medication to attain normotensive BP measurements.

Analysis of Patient Data

Following completion of the study we had collected 1108 patient samples with: (1) response data, and (2) genotype information for 120 SNPs. SNPs were different depending upon each drug class.

Linear Analysis

As a first step, a linear association analysis was performed to screen for “Golden SNPs”, single SNPs that could be used independently to predict response. Since hypertension is a complex disease involving many genes as detailed above, we did not expect to find any, however, these SNPs alone or in combination could be relevant to disease prediction in smaller subgroups of people.

To show the amount of linear correlation with response, we give χ² and Pearson's r coefficients and their significance. To review, the χ² coefficient gives a measurement of association. The null hypothesis is that two variables have no association. A high χ² value indicates that the null hypothesis is unlikely. The chi-square statistic is given by

$${χ}^{2} = {\sum }_{i , j} \frac{{( {N}_{i , j} - {n}_{i , j} )}^{2}}{{n}_{i , j}} .$$

In our case, we are measuring the association between phenotype and genotype. There are two phenotype values, response/non-response, and three-genotype variables, 2 homozygous combinations and one heterozygous combination. Here, Nᵢⱼ is the observed number of responses/non-responses for each genotype value, and nᵢⱼ is the number of responses/non-response in the null hypothesis.

χ² 's significance is given by Q (χ|ν), an incomplete gamma function, where ν is the degrees of freedom. Strictly speaking, Q is the probability that the sum of the squares of ν random normal variables of unit variance (and zero mean) will be greater than χ². Essentially, we are asking whether or not the sum of errors, N-n, are less than the sum of errors from an uncorrelated distribution of random variables. If Q is high, then it is likely that the errors of the uncorrelated distribution are greater than the errors in the data, indicating that the data is likely more correlated than some multi-variate normally distributed data set.

Hence, we then calculate Pearson's r, the linear correlation coefficient, for each genotype. Pearson's r gives a number between −1 and 1, with −1 indicating a high negative correlation, 1 a high positive correlation and 0 no correlation. In case a variable is constant (no genotypic variation), Pearson's r is undefined. We also supply the significance of the correlation, P, equal to 1−Q. P is the complimentary error function and it gives the probability that |r| should be larger than its observed value in the null hypothesis that the variable is uncorrelated with response/non-response. In other words, P is the probability that if the variable is uncorrelated then |r| would be larger than what is observed |r|. A small value of P indicates that the variable is significantly correlated with response/non-response.

In a preferred embodiment of the present invention, to enable higher predictive accuracy, one can use the top N SNP groups to train a committee network, described below, in a voting scheme. Basically N predictors of N sets of groups each give a “vote” to new, previously unseen examples presented to each predictor. The votes are added up and a final output is given based upon this “group vote”. This methodology yields hierarchical models with increased predictive accuracy.

Statistical method used for finding combinations of SNPs indicative of hypertension treatment

First, the SNPs were coded numerically as minor homozygous, heterozygous, and major homozygous, based on a frequency analysis of the complete data set. Then SNPs with zero variance were removed. A naïve subset of data was created by selecting at random 20% of the responder data and 20% of the non-responder data. This balanced data set was removed from the full data set and reserved for testing, while the remainder of the full data set was used to develop ten models. Each of the models were developed using a wrapped feature selection and model parameter estimation approach. The feature selection method used was SFFS, modified so that 10 independent feature sets are initialized and maintained through completion to create the 10 models. The machine learning method employed was kernel partial least squares with a polynomial order of 2 and a maximum of five latent variables used in the kernel. The cost function used during SFFS was the sum of the area under the receiver operator curve (ROC) and the specificity achieved by the model at the first sensitivity observed on the ROC curve greater than 0.93. Specifically, this cost function was evaluated for both a training set and a 5× cross-validation set maintained during training. It is to be noted that the naïve testing set was never used during the model building process. After termination of the SFFS procedure, the 10 feature sets were ranked based on the cost function score. The SNPs selected were assessed for HWD and for LD with each other. In the case of different SNP sets that were not statistically different from for the best overall performance, the set with the fewest number of SNPs in LD with each other, and the greatest number of SNPs in HWD was selected as the primary SNP set and corresponding model to evaluate on the naïve data set.

Results-Beta Blockers:

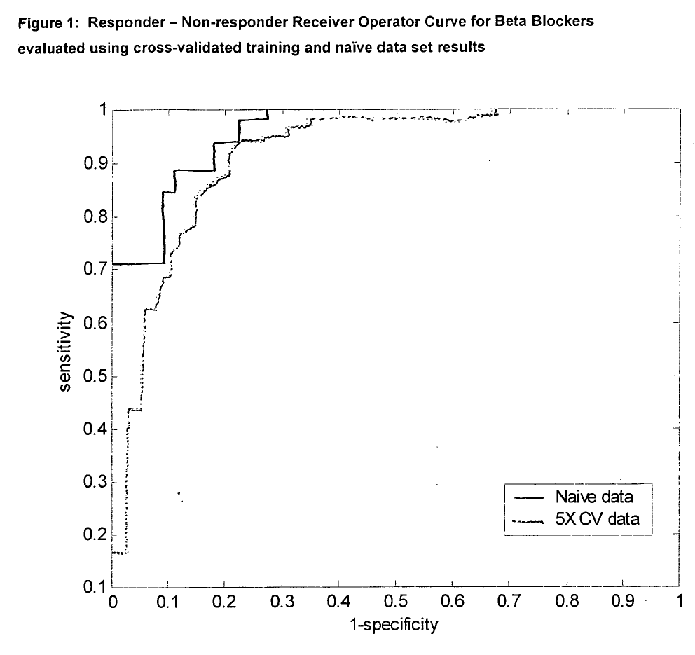

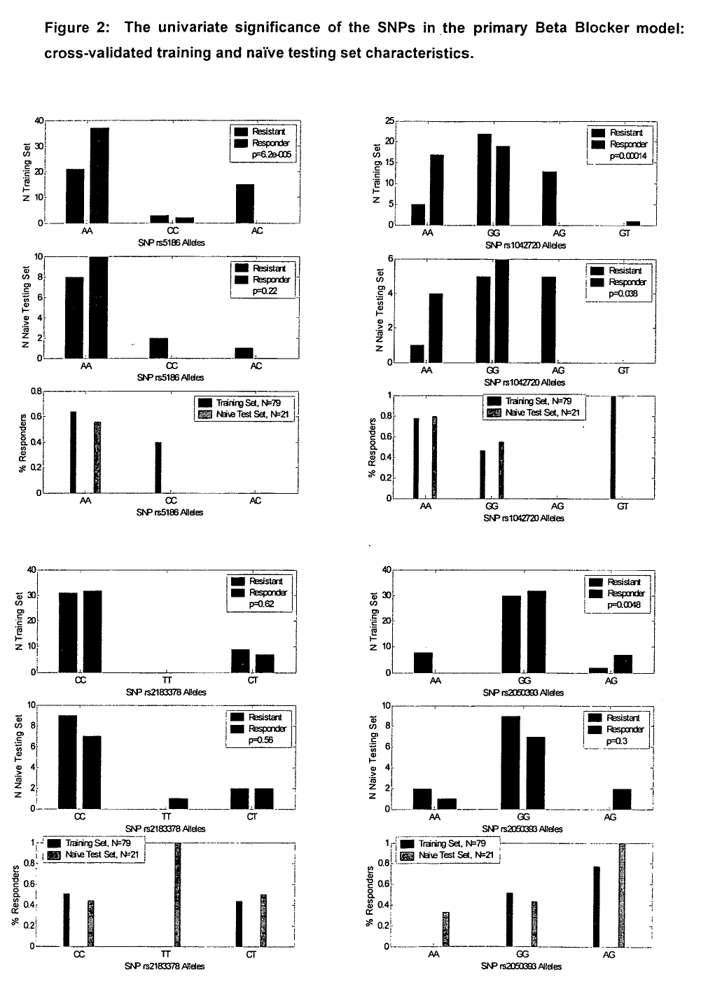

The identities of the selected SNPS, and their characteristics are given in Table 3. The ROC curves for the 5× cross-validation data and the naïve data set results using the primary model are shown in FIG. 1. The maximum product of sensitivity and specificity, and the sensitivity, specificity, and diagnostic odds ratio (DOR) at the maximum product operation point on the ROC are given in Table 4 for reference. The univariate statistically significance for each of the SNPs in the model with respect to response/non-response outcome are shown in FIG. 2 along with the number and percentage of responders and the number of non-responders in both the model training set and the naïve testing set.

TABLE 3
Identity and characteristics of the SNP set in the primary Beta Blocker model

| | | | | | LD - SNP |
|---|---|---|---|---|---|
| SNP RS# | Gene-Protein | HWD | Position | Role | p value |
| rs5186 | AGTR1 Angiotensin II receptor, | NA | chr3: 149942686 | 3PRIME_UTR | No |
| | type 1 | | | | |
| rs1042720 | ADRB2 Beta-2 adrenergic | NA | chr5: 148187826 | Coding - Synonymous | No |
| | receptor | | | | |
| rs2183378 | ADRB1, Beta-1 adrenergic | No, p = 0.371 | chr10: 115798882 | intron | rs2050393 |
| | receptor | | | | p < 1e−15 |
| rs2050393 | ADRB1, Beta-1 adrenergic | UD | chr10: 115804942 | intron | No |
| | receptor | | | | |

TABLE 4
Selected characteristics of the Responder - Non-responder receiver operator curve for
the cross-validation set and the naïve testing set for Beta Blocker model.

| | | Max Product | Sens@max | Spec@max | DOR@max | | |
|---|---|---|---|---|---|---|---|
| Data Set | AUC(ROC) | Sens*Spec | product | product | product | N | N Resp |
| Naïve | 0.96 | 0.79 | 0.89 | 0.89 | 62 | 21 | 10 |
| 5× CV | 0.91 | 0.73 | 0.94 | 0.78 | 52 | 80 | 40 |

### EXAMPLE II

Results-Calcium Channel Inhibitors:

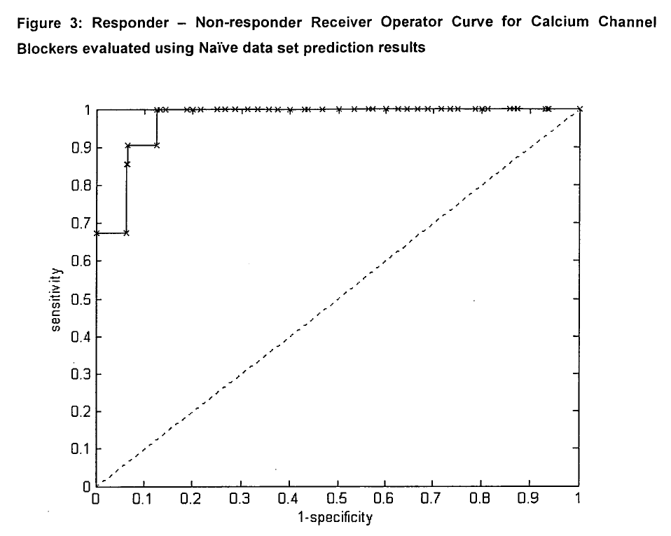

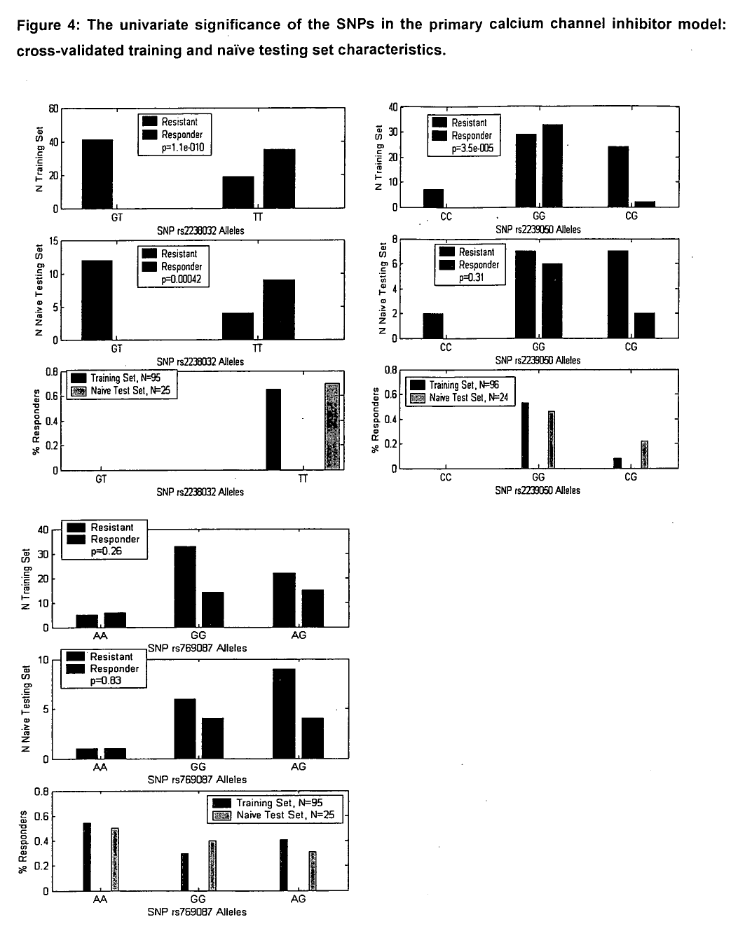

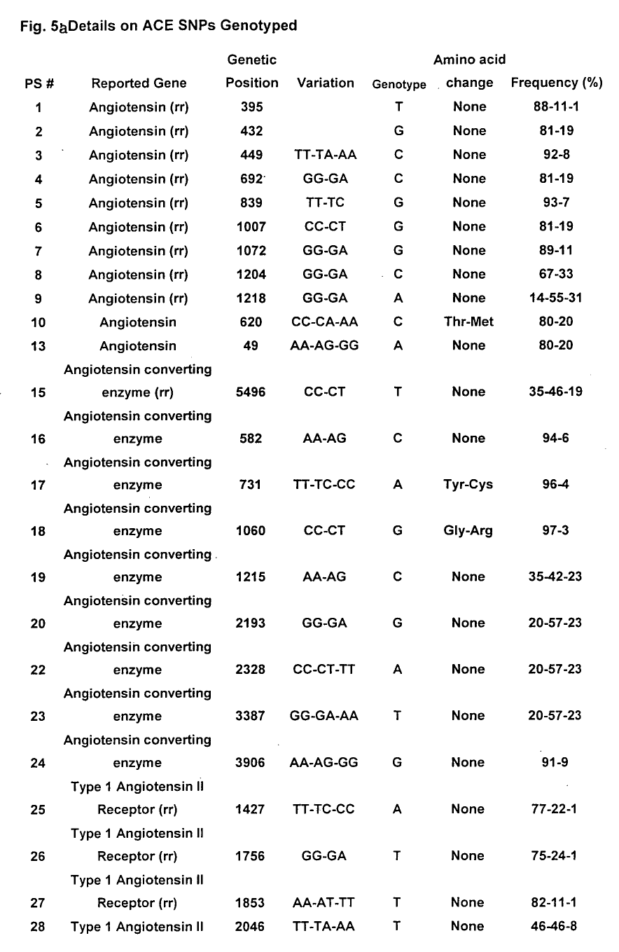

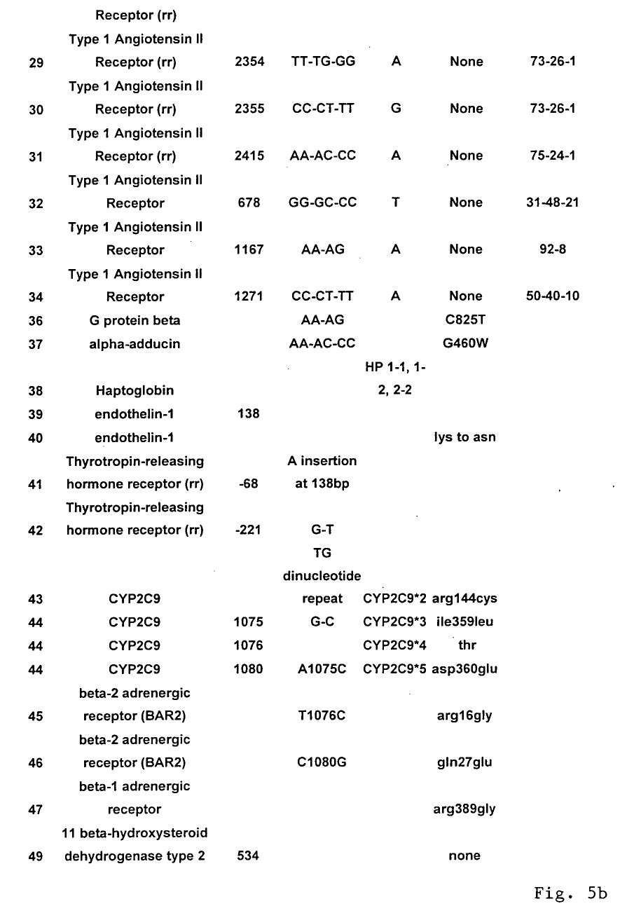

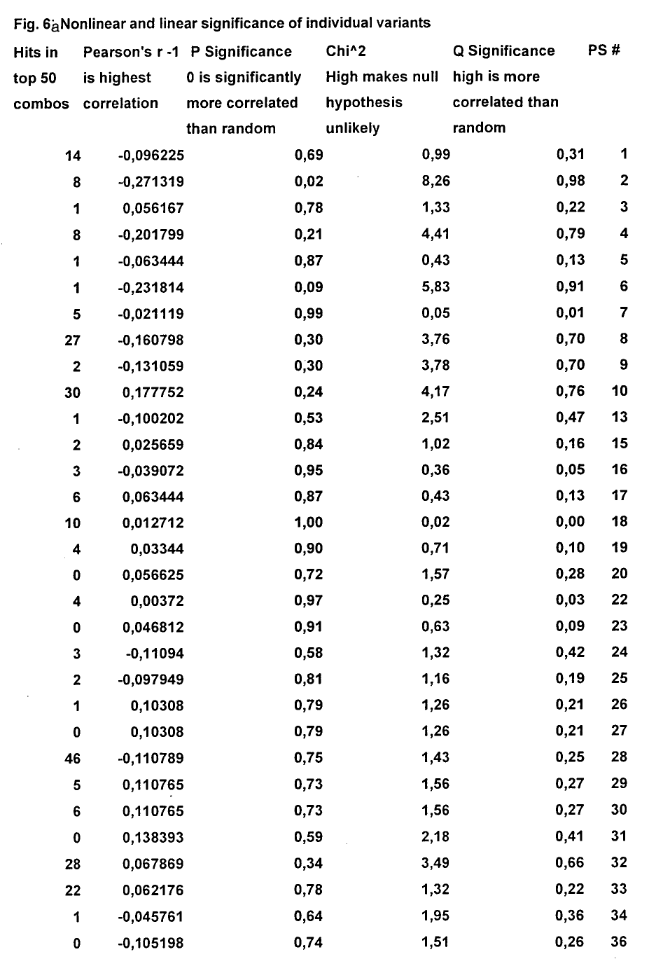

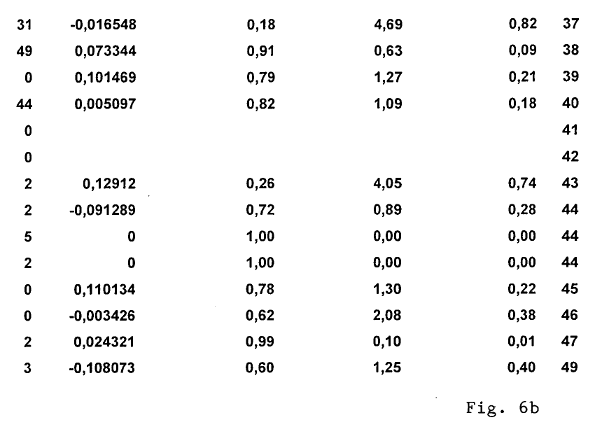

The identities of the selected SNPs, and their characteristics are given in Table 5. The ROC curves for naïve data set results using the primary model are shown in FIG. 3. The maximum product of sensitivity and specificity, and the sensitivity, specificity, and diagnostic odds ratio (DOR) at the maximum product operation point on the ROC are given in Table 6 for reference. The univariate statistical significance for each of the SNPs in the model with respect to response/non-response outcome are shown in FIG. 5 along with the number and percentage of responders and the number of non-responders in both the model training set and the naïve testing set.

TABLE 5
Identity and characteristics of the SNP set in the primary calcium channel inhibitor model

| | | | | | LD - S [symbol] |
|---|---|---|---|---|---|
| SNP RS# | Gene-Protein | HWD | Position | Role | p value |
| rs2238032 | CACNA1C calcium channel, voltage- | 0.001 | chr12: 2092993—2092993 | Intron | rs22390 |
| | dependent, L type, alpha 1C subunit | | | | p = 0.01 |
| rs2239050 | CACNA1C calcium channel, voltage- | 0.001 | chr12: 2317675—2317675 | Intron | No |
| | dependent, L type, alpha 1C subunit | | | | |
| rs769087 | CACNA1C calcium channel, voltage- | 0.001 | chr12: 2214905—2214905 | Intron | No |
| | dependent, L type, alpha 1C subunit | | | | |

TABLE 6
Selected characteristics of the Responder - Non-responder receiver operator curve for the
cross-validation set and the naïve testing set for Calcium Channel model.

| | | Max Product | Sens@max | Spec@max | DOR@max | N | N |
|---|---|---|---|---|---|---|---|
| Data Set | AUC(ROC) | Sens*Spec | product | product | product | Subjects | Responders |
| Naïve | 0.97 | 0.88 | 1 | 0.88 | >200 | 24 | 8 |
| 5× CV | 0.94 | 0.82 | 1 | 0.82 | >200 | 95 | 35 |

Diagnostic Detection of Hypertension Disease-Associated and Treatment-Relevant Mutations:

According to the present invention, base changes in the genes can be detected and used as a diagnostic for Hypertension. A variety of techniques are available for isolating DNA and RNA and for detecting mutations in the isolated AGT, ACE, AGTR1, CACNA1C, GPB, EDN1, EDN2, alpha-adducin, haptoglobin, CYP2C9, RGS2, ADRA1a, 11betaHSD2, ADRA1b, ADRA2A, ADRAB1, ADRAB2, REN, APOA, APOB, CETP, LIPC, EDNRB, or ENOS gene(s).

A number of sample preparation methods are available for isolating DNA and RNA from patient blood samples. For example, the DNA from a blood sample is obtained by cell lysis following alkali treatment. Often, there are multiple copies of RNA message per DNA. Accordingly, it is useful from the standpoint of detection sensitivity to have a sample preparation protocol which isolates both forms of nucleic acid. Total nucleic acid may be isolated by guanidium isothiocyanate/phenol-chloroform extraction, or by proteinase K/phenol-chloroform treatment. Commercially available sample preparation methods such as those from Qiagen Inc. (Chatsworth, Calif.) can also be utilized.

As discussed more fully hereinbelow, hybridization with one or more labelled probes containing complements of the variant sequences enables detection of the Hypertension mutations. Since each Hypertension patient can be heteroplasmic (possessing both the Hypertension mutation and the normal sequence) a quantitative or semi-quantitative measure (depending on the detection method) of such heteroplasmy can be obtained by comparing the amount of signal from the Hypertension probe to the amount from the Hypertension.sup.—(normal or wild-type) probe.

A variety of techniques, as discussed more fully hereinbelow, are available for detecting the specific mutations in the AGT, ACE, AGTR1, CACNA1C, GPB, EDN1, EDN2, alpha-adducin, haptoglobin, CYP2C9, RGS2, ADRA1a, 11betaHSD2, ADRA1b, ADRA2A, ADRAB1, ADRAB2, REN, APOA, APOB, CETP, LIPC, EDNRB, or ENOS gene(s). The detection methods include, for example, cloning and sequencing, ligation of oligonucleotides, use of the polymerase chain reaction and variations thereof, use of single nucleotide primer-guided extension assays, hybridization techniques using target-specific oligonucleotides and sandwich hybridization methods.

Cloning and sequencing of the AGT, ACE, AGTR1, CACNA1C, GPB, EDN1, EDN2, alpha-adducin, haptoglobin, CYP2C9, RGS2, ADRA1a, 11betaHSD2, ADRA1b, ADRA2A, ADRAB1, ADRAB2, REN, APOA, APOB, CETP, LIPC, EDNRB, or ENOS gene(s) can serve to detect Hypertension mutations in patient samples. Sequencing can be carried out with commercially available automated sequencers utilizing fluorescently labelled primers. An alternate sequencing strategy is the “sequencing by hybridization” method using high density oligonucleotide arrays on silicon chips (Fodor et al., Nature 364:555-556 (1993); Pease et al., Proc. Natl. Acad. Sci. USA, 91:5022-5026 (1994). For example, fluorescently-labelled target nucleic acid generated, for example from PCR amplification of the target genes using fluorescently labeled primers, are hybridized with a chip containing a set of short oligonucleotides which probe regions of complementarity with the target sequence. The resulting hybridization patterns are useful for reassembling the original target DNA sequence.

Mutational analysis can also be carried out by methods based on ligation of oligonucleotide sequences which anneal immediately adjacent to each other on a target DNA or RNA molecule (Wu and Wallace, Genomics 4:560-569 (1989); Landren et al., Science 241:1077-1080 (1988); Nickerson et al., Proc. Natl. Acad. Sci. 87:8923-8927 (1990); Barany, F., Proc. Natl. Acad. Sci. 88:189-193 (1991)). Ligase-mediated covalent attachment occurs only when the oligonucleotides are correctly base-paired. The Ligase Chain Reaction (LCR), which utilizes the thermostable Taq ligase for target amplification, is particularly useful for interrogating Hypertension mutation loci. The elevated reaction temperatures permits the ligation reaction to be conducted with high stringency (Barany, F., PCR Methods and Applications 1:5-16 (1991)).

Analysis of point mutations in DNA can also be carried out by using the polymerase chain reaction (PCR) and variations thereof. Mismatches can be detected by competitive oligonucleotide priming under hybridization conditions where binding of the perfectly matched primer is favored (Gibbs et al., Nucl. Acids. Res. 17:2437-2448 (1989)). In the amplification refractory mutation system technique (ARMS), primers are designed to have perfect matches or mismatches with target sequences either internal or at the 3′ residue (Newton et al., Nucl. Acids. Res. 17:2503-2516 (1989)). Under appropriate conditions, only the perfectly annealed oligonucleotide functions as a primer for the PCR reaction, thus providing a method of discrimination between normal and mutant (Hypertension) sequences.

Genotyping analysis of the Aldosterone synthase CYP11B2, Angiotensin converting enzyme ACE, CYP2C9, alpha-adducin, Angiotensinogen AGT, Angiotensin II type 1 receptor AGTR1, Angiotensin II type 2 receptor AGTR2, Mineralocorticoid receptor MLR, RGS2, Renin REN, Adrenergic [symbol] 1a receptor ADRA1a, Adrenergic [symbol] 1b receptor ADRA1b, Adrenergic [symbol] 2 receptor ADRA2A, Adrenergic_{—}1 receptor ADRB1, Adrenergic _{—}2 receptor ADRB2, Adrenergic _{—}3 receptor ADRB3, Endothelin receptor type A EDNRA, Endothelin receptor type B EDNRB, Endothelial nitric oxide synthase ENOS, Apolipoprotein A APOA, Apolipoprotein B APOB, Apolipoprotein E APOE, Lipase hepatic LIPC, Haptoglobin and Cholesteryl ester transfer protein CETP genes can also be carried out using single nucleotide primer-guided extension assays, where the specific incorporation of the correct base is provided by the high fidelity of the DNA polymerase (Syvanen et al., Genomics 8:684-692 (1990); Kuppuswamy et al., Proc. Natl. Acad. Sci. USA. 88:1143-1147 (1991)). Another primer extension assay, which allows for the quantification of heteroplasmy by simultaneously interrogating both wild-type and mutant nucleotides, is disclosed in a pending U.S. patent application entitled, “Multiplexed Primer Extension Methods”, naming Eoin Fahy and Soumitra Ghosh as inventors, filed on Mar. 24, 1995, Ser. No. 08/410,658, the disclosure of which is incorporated by reference.

Detection of single base mutations in target nucleic acids can be conveniently accomplished by differential hybridization techniques using target-specific oligonucleotides (Suggs et al., Proc. Natl. Acad. Sci. 78:6613-6617 (1981); Conner et al., Proc. Natl. Acad. Sci. 80:278-282 (1983); Saiki et al., Proc. Natl. Acad. Sci. 86:6230-6234 (1989)). For example, mutations are diagnosed on the basis of the higher thermal stability of the perfectly matched probes as compared to the mismatched probes. The hybridization reactions may be carried out in a filter-based format, in which the target nucleic acids are immobilized on nitrocellulose or nylon membranes and probed with oligonucleotide probes. Any of the known hybridization formats may be used, including Southern blots, slot blots, “reverse” dot blots, solution hybridization, solid support based sandwich hybridization, bead-based, silicon chip-based and microtiter well-based hybridization formats.

An alternative strategy involves detection of the AGT, ACE, AGTR1, CACNA1C, GPB, EDN1, EDN2, alpha-adducin, haptoglobin, CYP2C9, RGS2, ADRA1a, 11betaHSD2, ADRA1b, ADRA2A, ADRAB1, ADRAB2, REN, APOA, APOB, CETP, LIPC, EDNRB, or ENOS gene(s) by sandwich hybridization methods. In this strategy, the mutant and wild-type (normal) target nucleic acids are separated from non-homologous DNA/RNA using a common capture oligonucleotide immobilized on a solid support and detected by specific oligonucleotide probes tagged with reporter labels. The capture oligonucleotides can be immobilized on microtitre plate wells or on beads (Gingeras et al., J. Infect. Dis. 164:1066-1074 (1991); Richman et al., Proc. Natl. Acad. Sci. 88:11241-11245 (1991)).

While radio-isotopic labeled detection oligonucleotide probes are highly sensitive, non-isotopic labels are preferred due to concerns about handling and disposal of radioactivity. A number of strategies are available for detecting target nucleic acids by non-isotopic means (Matthews et al., Anal. Biochem., 169:1-25 (1988)). The non-isotopic detection method may be direct or indirect.

The indirect detection process is generally where the oligonucleotide probe is covalently labelled with a hapten or ligand such as digoxigenin (DIG) or biotin. Following the hybridization step, the target-probe duplex is detected by an antibody- or streptavidin-enzyme complex. Enzymes commonly used in DNA diagnostics are horseradish peroxidase and alkaline phosphatase. One particular indirect method, the Genius.™. detection system (Boehringer Mannheim) is especially useful for mutational analysis of the AGT, ACE, AGTR1, CACNA1C, GPB, EDN1, EDN2, alpha-adducin, haptoglobin, CYP2C9, RGS2, ADRA1a, 11betaHSD2, ADRA1b, ADRA2A, ADRAB1, ADRAB2, REN, APOA, APOB, CETP, LIPC, EDNRB, or ENOS gene(s). This indirect method uses digoxigenin as the tag for the oligonucleotide probe and is detected by an anti-digoxigenin-antibody-alkaline phosphatase conjugate.

Direct detection methods include the use of fluorophor-labeled oligonucleotides, lanthanide chelate-labeled oligonucleotides or oligonucleotide-enzyme conjugates. Examples of fluorophor labels are fluorescein, rhodamine and phthalocyanine dyes. Examples of lanthanide chelates include complexes of Eu.sup.3+ and Tb.sup.3+. Directly labeled oligonucleotide-enzyme conjugates are preferred for detecting point mutations when using target-specific oligonucleotides as they provide very high sensitivities of detection.

Oligonucleotide-enzyme conjugates can be prepared by a number of methods (Jablonski et al., Nucl. Acids Res., 14:6115-6128 (1986); Li et al., Nucl. Acids Res. 15:5275-5287 (1987); Ghosh et al., Bioconjugate Chem. 1:71-76 (1990)), and alkaline phosphatase is the enzyme of choice for obtaining high sensitivities of detection. The detection of target nucleic acids using these conjugates can be carried out by filter hybridization methods or by bead-based sandwich hybridization (Ishii et al., Bioconjugate Chemistry 4:34-41 (1993)).

Detection of the probe label may be accomplished by the following approaches. For radioisotopes, detection is by autoradiography, scintillation counting or phosphor imaging. For hapten or biotin labels, detection is with antibody or streptavidin bound to a reporter enzyme such as horseradish peroxidase or alkaline phosphatase, which is then detected by enzymatic means. For fluorophor or lanthanide-chelate labels, fluorescent signals may be measured with spectrofluorimeters with or without time-resolved mode or using automated microtitre plate readers. With enzyme labels, detection is by color or dye deposition (p-nitropheny phosphate or 5-bromo-4-chloro-3-indolyl phosphate/nitroblue tetrazolium for alkaline phosphatase and 3,3′-diaminobenzidine-NiCl.sub.2 for horseradish peroxidase), fluorescence (e.g., 4-methyl umbelliferyl phosphate for alkaline phosphatase) or chemiluminescence (the alkaline phosphatase dioxetane substrates LumiPhos 530 from Lumigen Inc., Detroit Mich. or AMPPD and CSPD from Tropix, Inc.). Chemiluminescent detection may be carried out with X-ray or polaroid film or by using single photon counting luminometers. This is the preferred detection format for alkaline phosphatase labelled probes.

The oligonucleotide probes for detection preferably range in size between 10 and 100 bases, more preferably between 15 and 30 bases in length. Examples of such nucleotide probes are found below in Tables 4 and 5. Tables 5 and 6 provide representative sequences of probes for detecting mutations in AGT, ACE, AGTR1, CACNA1C, GPB, EDN1, EDN2, alpha-adducin, haptoglobin, CYP2C9, RGS2, ADRA1a, 11betaHSD2, ADRA1b, ADRA2A, ADRAB1, ADRAB2, REN, APOA, APOB, CETP, LIPC, EDNRB, or ENOS gene(s) and representative antisense sequences. In order to obtain the required target discrimination using the detection oligonucleotide probes, the hybridization reactions are preferably run between 20.degree. C. and 60.degree. C., and more preferably between 30.degree. C. and 55.degree. C. As known to those skilled in the art, optimal discrimination between perfect and mismatched duplexes can be obtained by manipulating the temperature and/or salt concentrations or inclusion of formamide in the stringency washes.

As an alternative to detection of mutations in the nucleic acids associated with the AGT, ACE, AGTR1, CACNA1C, GPB, EDN1, EDN2, alpha-adducin, haptoglobin, CYP2C9, RGS2, ADRA1a, 11betaHSD2, ADRA1b, ADRA2A, ADRAB1, ADRAB2, REN, APOA, APOB, CETP, LIPC, EDNRB, or ENOS gene(s), it is also possible to analyze the protein products of the AGT, ACE, AGTR1, CACNA1C, GPB, EDN1, EDN2, alpha-adducin, haptoglobin, CYP2C9, RGS2, ADRA1a, 11betaHSD2, ADRA1b, ADRA2A, ADRAB1, ADRAB2, REN, APOA, APOB, CETP, LIPC, EDNRB, or ENOS gene(s). In particular, point mutations in these genes are expected to alter the structure of the proteins for which these gene encode. These altered proteins (variant polypeptides) can be isolated and used to prepare antisera and monoclonal antibodies that specifically detect the products of the mutated genes and not those of non-mutated or wild-type genes. Mutated gene products also can be used to immunize animals for the production of polyclonal antibodies. Recombinantly produced peptides can also be used to generate polyclonal antibodies. These peptides may represent small fragments of gene products produced by expressing regions of the mitochondrial genome containing point mutations.

More particularly, variant polypeptides from point mutations in said genes can be used to immunize an animal for the production of polyclonal antiserum. For example, a recombinantly produced fragment of a variant polypeptide can be injected into a mouse along with an adjuvant so as to generate an immune response. Murine immunoglobulins which bind the recombinant fragment with a binding affinity of at least 1.times.10.sup.7 M.sup.−1 can be harvested from the immunized mouse as an antiserum, and may be further purified by affinity chromatography or other means. Additionally, spleen cells are harvested from the mouse and fused to myeloma cells to produce a bank of antibody-secreting hybridoma cells. The bank of hybridomas can be screened for clones that secrete immunoglobulins which bind the recombinantly produced fragment with an affinity of at least 1.times.10.sup.6 M.sup.−1. More specifically, immunoglobulins that selectively bind to the variant polypeptides but poorly or not at all to wild-type polypeptides are selected, either by pre-absorption with wild-type proteins or by screening of hybridoma cell lines for specific idiotypes that bind the variant, but not wild-type, polypeptides.

Nucleic acid sequences capable of ultimately expressing the desired variant polypeptides can be formed from a variety of different polynucleotides (genomic or cDNA, RNA, synthetic oligonucleotides, etc.) as well as by a variety of different techniques.

The DNA sequences can be expressed in hosts after the sequences have been operably linked to (i.e., positioned to ensure the functioning of) an expression control sequence. These expression vectors are typically replicable in the host organisms either as episomes or as an integral part of the host chromosomal DNA. Commonly, expression vectors can contain selection markers (e.g., markers based on tetracyclinic resistance or hygromycin resistance) to permit detection and/or selection of those cells transformed with the desired DNA sequences. Further details can be found in U.S. Pat. No. 4,704,362.

Polynucleotides encoding a variant polypeptide may include sequences that facilitate transcription (expression sequences) and translation of the coding sequences such that the encoded polypeptide product is produced. Construction of such polynucleotides is well known in the art. For example, such polynucleotides can include a promoter, a transcription termination site (polyadenylation site in eukaryotic expression hosts), a ribosome binding site, and, optionally, an enhancer for use in eukaryotic expression hosts, and, optionally, sequences necessary for replication of a vector.

E. coli is one prokaryotic host useful particularly for cloning DNA sequences of the present invention. Other microbial hosts suitable for use include bacilli, such as Bacillus subtilus, and other enterobacteriaceae, such as Salmonella, Serratia, and various Pseudomonas species. In these prokaryotic hosts one can also make expression vectors, which will typically contain expression control sequences compatible with the host cell (e.g., an origin of replication). In addition, any number of a variety of well-known promoters will be present, such as the lactose promoter system, a tryptophan (Trp) promoter system, a beta-lactamase promoter system, or a promoter system from phage lambda. The promoters will typically control expression, optionally with an operator sequence, and have ribosome binding site sequences, for example, for initiating and completing transcription and translation.

Other microbes, such as yeast, may also be used for expression. Saccharomyces can be a suitable host, with suitable vectors having expression control sequences, such as promoters, including 3-phosphoglycerate kinase or other glycolytic enzymes, and an origin of replication, termination sequences, etc. as desired.

In addition to microorganisms, mammalian tissue cell culture may also be used to express and produce the polypeptides of the present invention. Eukaryotic cells are actually preferred, because a number of suitable host cell lines capable of secreting intact human proteins have been developed in the art, and include the CHO cell lines, various COS cell lines, HeLa cells, myeloma cell lines, Jurkat cells, and so forth. Expression vectors for these cells can include expression control sequences, such as an origin of replication, a promoter, an enhancer, an necessary information processing sites, such as ribosome binding sites, RNA splice sites, polyadenylation sites, and transcriptional terminator sequences. Preferred expression control sequences are promoters derived from immunoglobulin genes, SV40, Adenovirus, Bovine Papilloma Virus, and so forth. The vectors containing the DNA segments of interest (e.g., polypeptides encoding a variant polypeptide) can be transferred into the host cell by well-known methods, which vary depending on the type of cellular host. For example, calcium chloride transfection is commonly utilized for prokaryotic cells, whereas calcium phosphate treatment or electroporation may be used for other cellular hosts.

The method lends itself readily to the formulation of test kits for use in diagnosis. Such a kit would comprise a carrier compartmentalized to receive in close confinement one or more containers wherein a first container may contain suitably labeled DNA or immunological probes. Other containers may contain reagents useful in the localization of the labeled probes, such as enzyme substrates. Still other containers may contain restriction enzymes, buffers etc., together with instructions for use.

Therapeutic Treatment of Hypertension:

Suppressing the effects of the mutations through antisense or short interfering (siRNA) technology provides an effective therapy for Hypertension. Much is known about ‘antisense’ or siRNA therapies targeting messenger RNA (mRNA) or nuclear DNA. Hien et al., Biochem. Biophys. Acta 1049:99-125 (1990). The diagnostic test of the present invention is useful for determining which of the specific Hypertension mutations exist in a particular Hypertension patient; this allows for “custom” treatment of the patient with antisense or siRNA oligonucleotides only for the detected mutations. This patient-specific antisense therapy is also novel, and minimizes the exposure of the patient to any unnecessary antisense or siRNA therapeutic treatment. As used herein, an “antisense” oligonucleotide is one that base pairs with single stranded DNA or RNA by Watson-Crick base pairing and with duplex target DNA via Hoogsteen hydrogen bonds.

RNA interference refers to the process of sequence-specific post-transcriptional gene silencing in animals mediated by short interfering RNAs (siRNAs). The process of post-transcriptional gene silencing is an evolutionarily conserved cellular defense mechanism believed to prevent the expression of foreign genes. Such protection from foreign gene expression may have evolved in response to the production of double-stranded RNAs (dsRNAs) derived from viral infection, or from the random integration of transposon elements into the host genome. The presence of dsRNA in cells triggers the RNAi response through a mechanism that has yet to be fully characterized. The presence of long dsRNAs in cells stimulates the activity of a ribonuclease III enzyme referred to as dicer.

Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also uses an endonuclease complex, commonly referred to as an RNA-induced silencing complex (RISC). RISC mediates cleavage of single-stranded RNA having sequence complementarity to the antisense strand associated with the complex. RNA interference (RNAi) has been harnessed in laboratory cell culture systems and widely applied to identify the function of genes and their respective proteins. Moreover, RNAi holds promise for the development of a brand new class of drugs, capable of turning off disease-causing genes. These drugs could have specificity and potential applications in a number of therapeutic indications. For more detail see U.S. Pat. No. 5,854,038 entitled ‘Localization Of Therapeutic Agent In A Cell In Vitro’.

Another preferred methodology uses DNA directed RNA interference (ddRNAi). ddRNAi relies on RNA polymerase III (Pol III) promoters (e.g. U6 or H1) for the expression of siRNA target sequences that have been transfected in mammalian cells.

Pol III directs the synthesis of small RNA transcripts whose 3′ ends are defined by termination within a stretch of 4-5 thymidines. These characteristics allow for the use of DNA templates to synthesize, in vivo, small RNA duplexes that are structurally equivalent to active siRNAs synthesized in vitro.

siRNA/RISC duplexes form in the cell and lead to the degradation of the target mRNA. siRNA target sequences then can be introduced into the cell by a ddRNAi expression cassette or by being cloned in a siRNA expression vector. For more detail see Gou, D. et al. (2003) Gene Silencing in mammalian cells by PCR-based short hairpin RNA FEBS 548, 113-118.

The destructive effect of the Hypertension mutations in AGT, ACE, AGTR1, CACNA1C, GPB, EDN1, EDN2, alpha-adducin, haptoglobin, CYP2C9, RGS2, ADRA1a, 11betaHSD2, ADRA1b, ADRA2A, ADRAB1, ADRAB2, REN, APOA, APOB, CETP, LIPC, EDNRB, or ENOS gene(s) is preferably reduced or eliminated using antisense or siRNA oligonucleotide agents. Such antisense agents target DNA, by triplex formation with double-stranded DNA, by duplex formation with single-stranded DNA during transcription, or both. In a preferred embodiment, antisense agents target messenger RNA coding for the mutated AGT, ACE, AGTR1, CACNA1C, GPB, EDN1, EDN2, alpha-adducin, haptoglobin, CYP2C9, RGS2, ADRA1a, 11betaHSD2, ADRA1b, ADRA2A, ADRAB1, ADRAB2, REN, APOA, APOB, CETP, LIPC, EDNRB, or ENOS gene(s). Since the sequences of both the DNA and the mRNA are the same, it is not necessary to determine accurately the precise target to account for the desired effect. Procedures for inhibiting gene expression in cell culture and in vivo can be found, for example, in C. F. Bennett, et al. J. Liposome Res., 3:85 (1993) and C. Wahlestedt, et al. Nature, 363:260 (1993).

Antisense oligonucleotide therapeutic agents demonstrate a high degree of pharmaceutical specificity. This allows the combination of two or more antisense therapeutics at the same time, without increased cytotoxic effects. Thus, when a patient is diagnosed as having two or more Hypertension mutations in AGT, ACE, AGTR1, CACNA1C, GPB, EDN1, EDN2, alpha-adducin, haptoglobin, CYP2C9, RGS2, ADRA1a, 11betaHSD2, ADRA1b, ADRA2A, ADRAB1, ADRAB2, REN, APOA, APOB, CETP, LIPC, EDNRB, or ENOS gene(s), the therapy is preferably tailored to treat the multiple mutations simultaneously. When combined with the present diagnostic test, this approach to “patient-specific therapy” results in treatment restricted to the specific mutations detected in a patient. This patient-specific therapy circumvents the need for ‘broad spectrum’ antisense treatment using all possible mutations. The end result is less costly treatment, with less chance for toxic side effects.

One method to inhibit the synthesis of proteins is through the use of antisense or triplex oligonucleotides, analogues or expression constructs. These methods entail introducing into the cell a nucleic acid sufficiently complementary in sequence so as to specifically hybridize to the target gene or to mRNA. In the event that the gene is targeted, these methods can be extremely efficient since only a few copies per cell are required to achieve complete inhibition. Antisense methodology inhibits the normal processing, translation or half-life of the target message. Such methods are well known to one skilled in the art.

Antisense and triplex methods generally involve the treatment of cells or tissues with a relatively short oligonucleotide, although longer sequences can be used to achieve inhibition. The oligonucleotide can be either deoxyribo- or ribonucleic acid and must be of sufficient length to form a stable duplex or triplex with the target RNA or DNA at physiological temperatures and salt concentrations. It should also be sufficiently complementary or sequence specific to specifically hybridize to the target nucleic acid. Oligonucleotide lengths sufficient to achieve this specificity are preferably about 10 to 60 nucleotides long, more preferably about 10 to 20 nucleotides long. However, hybridization specificity is not only influenced by length and physiological conditions but may also be influenced by such factors as GC content and the primary sequence of the oligonucleotide. Such principles are well known in the art and can be routinely determined by one who is skilled in the art.

As an example, many of the oligonucleotide sequences used in connection with probes can also be used as antisense agents, directed to either the DNA or resultant messenger RNA.

A great range of antisense sequences can be designed for a given mutation. Oligonucleotide sequences can be easily designed by one of ordinary skill in the art to function as RNA and DNA antisense sequences for the mutant genes AGT, ACE, AGTR1, CACNA1C, GPB, EDN1, EDN2, alpha-adducin, haptoglobin, CYP2C9, RGS2, ADRA1a, 11betaHSD2, ADRA1b, ADRA2A, ADRAB1, ADRAB2, REN, APOA, APOB, CETP, LIPC, EDNRB, or ENOS gene(s).

As can be seen, permutations can be generated for a selected mutant antigene by truncating the 5′ end, truncating the 3′ end, extending the 5′ end, or extending the 3′ end. Both light chain and heavy chain mtDNA can be targeted. Other variations such as truncating the 5′ end and truncating the 3′ end, extending the 5′ end and extending the 3′ end, and truncating the 5′ end and extending the 3′ end, extending the 5′ end and truncating the 3′ end, and so forth are possible.

The composition of the antisense or triplex oligonucleotides can also influence the efficiency of inhibition. For example, it is preferable to use oligonucleotides that are resistant to degradation by the action of endogenous nucleases. Nuclease resistance will confer a longer in vivo half-life to the oligonucleotide thus increasing its efficacy and reducing the required dose. Greater efficacy may also be obtained by modifying the oligonucleotide so that it is more permeable to cell membranes. Such modifications are well known in the art and include the alteration of the negatively charged phosphate backbone bases, or modification of the sequences at the 5′ or 3′ terminus with agents such as intercalators and crosslinking molecules. Specific examples of such modifications include oligonucleotide analogs that contain methylphosphonate (Miller, P. S., Biotechnology, 2:358-362 (1991)), phosphorothioate (Stein, Science 261:1004-1011 (1993)) and phosphorodithioate linkages (Brill, W. K-D., J. Am. Chem. Soc., 111:2322 (1989)). Other types of linkages and modifications exist as well, such as a polyamide backbone in peptide nucleic acids (Nielson et al., Science 254:1497 (1991)), formacetal (Matteucci, M., Tetrahedron Lett. 31:2385-2388 (1990)) carbamate and morpholine linkages as well as others known to those skilled in the art. In addition to the specificity afforded by the antisense agents, the target RNA or genes can be irreversibly modified by incorporating reactive functional groups in these molecules which covalently link the target sequences e.g. by alkylation.

Recombinant methods known in the art can also be used to achieve the antisense or triplex inhibition of a target nucleic acid. For example, vectors containing antisense nucleic acids can be employed to express protein or antisense message to reduce the expression of the target nucleic acid and therefore its activity. Such vectors are known or can be constructed by those skilled in the art and should contain all expression elements necessary to achieve the desired transcription of the antisense or triplex sequences. Other beneficial characteristics can also be contained within the vectors such as mechanisms for recovery of the nucleic acids in a different form. Phagemids are a specific example of such beneficial vectors because they can be used either as plasmids or as bacteriophage vectors. Examples of other vectors include viruses, such as bacteriophages, baculoviruses and retroviruses, cosmids, plasmids, liposomes and other recombination vectors. The vectors can also contain elements for use in either procaryotic or eukaryotic host systems. One of ordinary skill in the art will know which host systems are compatible with a particular vector.

The vectors can be introduced into cells or tissues by any one of a variety of known methods within the art. Such methods are described for example in Sambrook et al., Molecular Cloning: A Laboratory Manual, Cold Spring Harbor Laboratory, New York (1992), which is hereby incorporated by reference, and in Ausubel et al., Current Protocols in Molecular Biology, John Wiley and Sons, Baltimore, Md. (1989), which is also hereby incorporated by reference. The methods include, for example, stable or transient transfection, lipofection, electroporation and infection with recombinant viral vectors. Introduction of nucleic acids by infection offers several advantages over the other listed methods which includes their use in both in vitro and in vivo settings. Higher efficiency can also be obtained due to their infectious nature. Moreover, viruses are very specialized and typically infect and propagate in specific cell types. Thus, their natural specificity can be used to target the antisense vectors to specific cell types in vivo or within a tissue or mixed culture of cells. Viral vectors can also be modified with specific receptors or ligands to alter target specificity through receptor mediated events.

A specific example of a viral vector for introducing and expressing antisense nucleic acids is the adenovirus derived vector Adenop53TX. This vector expresses a herpes virus thymidine kinase (TX) gene for either positive or negative selection and an expression cassette for desired recombinant sequences such as antisense sequences. This vector can be used to infect cells including most cancers of epithelial origin, glial cells and other cell types. This vector as well as others that exhibit similar desired functions can be used to treat a mixed population of cells to selectively express the antisense sequence of interest. A mixed population of cells can include, for example, in vitro or ex vivo culture of cells, a tissue or a human subject.

Additional features may be added to the vector to ensure its safety and/or enhance its therapeutic efficacy. Such features include, for example, markers that can be used to negatively select against cells infected with the recombinant virus. An example of such a negative selection marker is the TK gene described above that confers sensitivity to the antibiotic gancyclovir. Negative selection is therefore a means by which infection can be controlled because it provides inducible suicide through the addition of antibiotics. Such protection ensures that if, for example, mutations arise that produce mutant forms of the viral vector or antisense sequence, cellular transformation will not occur. Moreover, features that limit expression to particular cell types can also be included. Such features include, for example, promoter and expression elements that are specific for the desired cell type.

The foregoing and following description of the invention and the various embodiments is not intended to be limiting of the invention but rather is illustrative thereof. Those skilled in the art of molecular genetics can formulate further embodiments encompassed within the scope of the present invention.

### FURTHER ILLUSTRATIVE EXAMPLES

Definitions of Abbreviations:

1.times. SSC=150 mM sodium chloride, 15 mM sodium citrate, pH 6.5-8

SDS=sodium dodecyl sulfate

BSA=bovine serum albumin, fraction IV

probe=a labelled nucleic acid, generally a single-stranded oligonucleotide, which is complementary to the DNA target immobilized on the membrane. The probe may be labelled with radioisotopes (such as .sup.32P), haptens (such as digoxigenin), biotin, enzymes (such as alkaline phosphatase or horseradish peroxidase), fluorophores (such as fluorescein or Texas Red), or chemilumiphores (such as acridine).

PCR=polymerase chain reaction, as described by Erlich et al., Nature 331:461-462 (1988) hereby incorporated by reference.

### EXAMPLE III

Sequencing of AGT, ACE, AGTR1, CACNA1C, GPB, EDN1, EDN2, alpha-adducin, haptoglobin, CYP2C9, RGS2, ADRA1a, 11betaHSD2, ADRA1b, ADRA2A, ADRAB1, ADRAB2, REN, APOA, APOB, CETP, LIPC, EDNRB, or ENOS gene(s)

Plasmid DNA containing the AGT, ACE, AGTR1, CACNA1C, GPB, EDN1, EDN2, alpha-adducin, haptoglobin, CYP2C9, RGS2, ADRA1a, 11betaHSD2, ADRA1b, ADRA2A, ADRAB1, ADRAB2, REN, APOA, APOB, CETP, LIPC, EDNRB, or ENOS gene(s) gene inserts is isolated using the Plasmid Quik.™. Plasmid Purification Kit (Stratagene, San Diego, Calif.) or the Plasmid Kit (Qiagen, Chatsworth, Calif., Catalog #12145). Plasmid DNA is purified from 50 ml bacterial cultures. For the Stratagene protocol “Procedure for Midi Columns,” steps 10-12 of the kit protocol are replaced with a precipitation step using 2 volumes of 100% ethanol at −20.degree. C., centrifugation at 6,000.times. g for 15 minutes, a wash step using 80% ethanol and resuspension of the DNA sample in 100 ul TE buffer. DNA concentration is determined by horizontal agarose gel electrophoresis, or by UV absorption at 260 nm.

Sequencing reactions using double-stranded plasmid DNA are performed using the Sequenase Kit (United States Biochemical Corp., Cleveland, Ohio.; catalog #70770), the BaseStation T7 Kit (Millipore Corp.; catalog #MBBLSEQ01), the Vent Sequencing Kit (Millipore Corp; catalog #MBBLVEN01), the AmpliTaq Cycle Sequencing Kit (Perkin Elmer Corp.; catalog #N808-0110) and the Taq DNA Sequencing Kit (Boehringer Mannheim). The DNA sequences are detected by fluorescence using the BaseStation Automated DNA Sequencer (Millipore Corp.). For gene walking experiments, fluorescent oligonucleotide primers are synthesized on the Cyclone Plus DNA Synthesizer (Millipore Corp.) or the GeneAssembler DNA Synthesizer (Pharmacia LKB Biotechnology, Inc.) utilizing beta-cyanoethylphosphoramidite chemistry. Primer sequences are prepared from the published Cambridge sequences of the AGT, ACE, AGTR1, CACNA1C, GPB, EDN1, EDN2, alpha-adducin, haptoglobin, CYP2C9, RGS2, ADRA1a, 11betaHSD2, ADRA1b, ADRA2A, ADRAB1, ADRAB2, REN, APOA, APOB, CETP, LIPC, EDNRB, or ENOS gene(s) by using public reference sources such as http://www.snpperchip.org Primers are deprotected and purified as described above. DNA concentration is determined by UV absorption at 260 nm.

Sequencing reactions are performed according to manufacturer's instructions except for the following modification: 1) the reactions are terminated and reduced in volume by heating the samples without capping to 94.degree. C. for 5 minutes, after which 4 .mu.l of stop dye (3 mg/ml dextran blue, 95%-99% formamide; as formulated by Millipore Corp.) are added; 2) the temperature cycles performed for the AmpliTaq Cycle Sequencing Kit reactions, the Vent Sequencing kit reactions, and the Taq Sequence Kit consist of one cycle at 95.degree. C. for 10 seconds, 30 cycles at 95.degree. C. for 20 seconds, at 44.degree. C. for 20 seconds and at 72.degree. C. for 20 seconds followed by a reduction in volume by heating without capping to 94.degree. C. for 5 minutes before adding 4 .mu.l of stop dye.

Electrophoresis and gel analysis are performed using the Biolmage and BaseStation Software provided by the manufacturer for the BaseStation Automated DNA Sequencer (Millipore Corp.). Sequencing gels are prepared according to the manufacturer's specifications. An average of ten different clones from each individual is sequenced. The resulting AGT, ACE, AGTR1, CACNA1C, GPB, EDN1, EDN2, alpha-adducin, haptoglobin, CYP2C9, RGS2, ADRA1a, 11betaHSD2, ADRA1b, ADRA2A, ADRAB1, ADRAB2, REN, APOA, APOB, CETP, LIPC, EDNRB, or ENOS gene(s) sequences are aligned and compared with published Cambridge sequences. Mutations in the derived sequence are noted and confirmed by resequencing the variant region.

As an alternative procedure for sequencing the AGT, ACE, AGTR1, CACNA1C, GPB, EDN1, EDN2, alpha-adducin, haptoglobin, CYP2C9, RGS2, ADRA1a, 11betaHSD2, ADRA1b, ADRA2A, ADRAB1, ADRAB2, REN, APOA, APOB, CETP, LIPC, EDNRB, or ENOS gene(s), plasmid DNA containing the AGT, ACE, AGTR1, CACNA1C, GPB, EDN1, EDN2, alpha-adducin, haptoglobin, CYP2C9, RGS2, ADRA1a, 11betaHSD2, ADRA1b, ADRA2A, ADRAB1, ADRAB2, REN, APOA, APOB, CETP, LIPC, EDNRB, or ENOS gene(s) gene inserts obtained is isolating the inserts using the Plasmid Quik.™. Plasmid Purification Kit with Midi Columns (Qiagen, Chatsworth, Calif.) Plasmid DNA is purified from 35 ml bacterial cultures. The isolated DNA is resuspended in 100 .mu.l TE buffer. DNA concentrations are determined by OD (260) absorption.

As an alternative method, sequencing reactions using double stranded plasmid DNA are performed using the Prism.™. Ready Reaction DyeDeoxy.™. Terminator Cycle Sequencing Kit (Applied Biosystems, Inc., Foster City, Calif.). The DNA sequences are detected by fluorescence using the ABI 373A Automated DNA Sequencer (Applied Biosystems, Inc., Foster City, Calif.). For gene walking experiments, oligonucleotide primers are synthesized on the ABI 394 DNA/RNA Synthesizer (Applied Biosystems, Inc., Foster City, Calif.) using standard beta-cyanoethylphosphoramidite chemistry. Primer sequences are prepared from the published Cambridge sequences of the AGT, ACE, AGTR1, CACNA1C, GPB, EDN1, EDN2, alpha-adducin, haptoglobin, CYP2C9, RGS2, ADRA1a, 11betaHSD2, ADRA1b, ADRA2A, ADRAB1, ADRAB2, REN, APOA, APOB, CETP, LIPC, EDNRB, or ENOS gene(s).

Sequencing reactions are performed according to the manufacturer's instructions. Electrophoresis and sequence analysis are performed using the ABI 373A Data Collection and Analysis Software and the Sequence Navigator Software (ABI, Foster City, Calif.). Sequencing gels are prepared according to the manufacturer's specifications. An average of ten different clones from each individual is sequenced. The resulting AGT, ACE, AGTR1, CACNA1C, GPB, EDN1, EDN2, alpha-adducin, haptoglobin, CYP2C9, RGS2, ADRA1a, 11betaHSD2, ADRA1b, ADRA2A, ADRAB1, ADRAB2, REN, APOA, APOB, CETP, LIPC, EDNRB, or ENOS gene(s) sequences are aligned and compared with the published Cambridge sequence. Mutations in the derived sequence are noted and confirmed by sequence of the complementary DNA strand.

Mutations in each AGT, ACE, AGTR1, CACNA1C, GPB, EDN1, EDN2, alpha-adducin, haptoglobin, CYP2C9, RGS2, ADRA1a, 11betaHSD2, ADRA1b, ADRA2A, ADRAB1, ADRAB2, REN, APOA, APOB, CETP, LIPC, EDNRB, or ENOS gene(s) for each individual are compiled. Comparisons of mutations between normal and Hypertension patients are made and an algorithm, described below, is used to provide diagnostic or prognostic prediction.

### EXAMPLE IV

Detection of AGT, ACE, AGTR1, CACNA1C, GPB, EDN1, EDN2, alpha-adducin, haptoglobin, CYP2C9, RGS2, ADRA1a, 11betaHSD2, ADRA1b, ADRA2A, ADRAB1, ADRAB2, REN, APOA, APOB, CETP, LIPC, EDNRB, or ENOS gene(s) Mutations by Hybridization Without Prior Amplification

This example illustrates taking test sample blood, blotting the DNA, and detecting by oligonucleotide hybridization in a dot blot format. This example uses two probes to determine the presence of the abnormal mutations of the AGT, ACE, AGTR1, CACNA1C, GPB, EDN1, EDN2, alpha-adducin, haptoglobin, CYP2C9, RGS2, ADRA1a, 11betaHSD2, ADRA1b, ADRA2A, ADRAB1, ADRAB2, REN, APOA, APOB, CETP, LIPC, EDNRB, or ENOS gene(s) in DNA of Hypertension patients. This example utilizes a dot-blot format for hybridization, however, other known hybridization formats, such as Southern blots, slot blots, “reverse” dot blots, solution hybridization, solid support based sandwich hybridization, bead-based, silicon chip-based and microtiter well-based hybridization formats can also be used.

Sample Preparation Extracts and Blotting of DNA onto Membranes:

Whole blood is taken from the patient. The blood is mixed with an equal volume of 0.5-1 N NaOH, and is incubated at ambient temperature for ten to twenty minutes to lyse cells, degrade proteins, and denature any DNA. The mixture is then blotted directly onto prewashed nylon membranes, in multiple aliquots. The membranes are rinsed in 10.times. SSC (1.5 M NaCl, 0.15 M Sodium Citrate, pH 7.0) for five minutes to neutralize the membrane, then rinsed for five minutes in 1.times. SSC. For storage, if any, membranes are air-dried and sealed. In preparation for hybridization, membranes are rinsed in 1.times. SSC, 1% SDS.

Alternatively, 1-10 mls of whole blood is fractionated by standard methods, and the white cell layer (“buffy coat”) is separated. The white cells are lysed, digested, and the DNA extracted by conventional methods (organic extraction, non-organic extraction, or solid phase). The DNA is quantitated by UV absorption or fluorescent dye techniques. Standardized amounts of DNA (0.1-5 .mu.g) are denatured in base, and blotted onto membranes. The membranes are then rinsed.

Alternative methods of preparing cellular DNA, such as isolation of DNA by mild cellular lysis and centrifugation, may also be used.

Hybridization and Detection:

For examples of synthesis, labelling, use, and detection of oligonucleotide probes, see “Oligonucleotides and Analogues: A Practical Approach”, F. Eckstein, ed., Oxford University Press (1992); and “Synthetic Chemistry of Oligonucleotides and Analogs”, S. Agrawal, ed., Humana Press (1993), which are incorporated herein by reference.

For detection and quantitation of the abnormal mutation, membranes containing duplicate samples of DNA are hybridized in parallel; one membrane is hybridized with the wild-type probe, the other with the Hypertension gene probe. Alternatively, the same membrane can be hybridized sequentially with both probes and the results compared.

For example, the membranes with immobilized DNA are hydrated briefly (10-60 minutes) in 1.times. SSC, 1% SDS, then prehybridized and blocked in 5.times. SSC, 1% SDS, 0.5% casein, for 30-60 minutes at hybridization temperature (35-60.degree. C., depending on which probe is used). Fresh hybridization solution containing probe (0.1-10 nM, ideally 2-3 nM) is added to the membrane, followed by hybridization at appropriate temperature for 15-60 minutes. The membrane is washed in 1.times. SSC, 11 SDS, 1-3 times at 45-60.degree. C. for 5-10 minutes each (depending on probe used), then 1-2 times in 1.times. SSC at ambient temperature. The hybridized probe is then detected by appropriate means.

The average proportion of Hypertension AGT, ACE, AGTR1, CACNA1C, GPB, EDN1, EDN2, alpha-adducin, haptoglobin, CYP2C9, RGS2, ADRA1a, 11betaHSD2, ADRA1b, ADRA2A, ADRAB1, ADRAB2, REN, APOA, APOB, CETP, LIPC, EDNRB, or ENOS gene(s) to wild-type gene(s) in the same patient can be determined by the ratio of the signal of the Hypertension probe to the normal probe. This is a semiquantitative measure of % heteroplasmy in the Hypertension patient and can be correlated to the severity of the disease.

The above and other probes for alteration and quantitation of wild-type and mutant DNA samples can be found at http://www.snpper.chip.org and typing in the RS numbers of the relevant mutations.

### EXAMPLE V

Detection of ABCB1, ABCB4, COMT, CRHR1, CRHR2, CRHBP, CYP2D6, CYP2D19, DRD2, DRD3,HRT2A, HTR3A, HTR3B, MAOA, MAOB, SLC6A3, OR SLC6A4 Mutations by Hybridization (Without Prior Amplification)

A. Slot-Blot Detection of RNA/DNA with .sup.32P Probes

This example illustrates detection of AGT, ACE, AGTR1, CACNA1C, GPB, EDN1, EDN2, alpha-adducin, haptoglobin, CYP2C9, RGS2, ADRA1a, 11betaHSD2, ADRA1b, ADRA2A, ADRAB1, ADRAB2, REN, APOA, APOB, CETP, LIPC, EDNRB, or ENOS gene(s) mutations by slot-blot detection of DNA with .sup.32p probes. The reagents are prepared as follows: 4.times. BP: 2% (w/v) Bovine serum albumin (BSA), 2% (w/v) polyvinylpyrrolidone (PVP, Mol. Wt.: 40,000) is dissolved in sterile H.sub.2O and filtered through 0.22-.mu. cellulose acetate membranes (Coming) and stored at −20.degree. C. in 50-ml conical tubes.

DNA is denatured by adding TE to the sample for a final volume of 90.mu.l. 10 .mu.l of 2 N NaOH is then added and the sample vortexed, incubated at 65.degree. C. for 30 minutes, and then put on ice. The sample is neutralized with 100 .mu.l of 2 M ammonium acetate.

A wet piece of nitrocellulose or nylon is cut to fit the slot-blot apparatus according to the manufacturer's directions, and the denatured samples are loaded. The nucleic acids are fixed to the filter by baking at 80.degree. C. under vacuum for 1 hr or exposing to UV light (254 nm). The filter is prehybridized for 10-30 minutes in 5 mls of 1.times. BP, 5.times. SSPE, 1% SDS at the temperature to be used for the hybridization incubation. For 15-30-base probes, the range of hybridization temperatures is between 35-60.degree. C. For shorter probes or probes with low G-C content, a lower temperature is used. At least 2.times.10.sup.6 cpm of detection oligonucleotide per ml of hybridization solution is added. The filter is double sealed in Scotchpak.™. heat sealable pouches (Kapak Corporation) and incubated for 90 min. The filter is washed 3 times at room temperature with 5-minute washes of 20.times. SSPE: 3M NaCl, 0.02M EDTA, 0.2 Sodium Phospate, pH 7.4, 1% SDS on a plafform shaker. For higher stringency, the filter can be washed once at the hybridization temperature in 1.times. SSPE, 1% SDS for 1 minute. Visualization is by autoradiography on Kodak XAR film at −70.degree. C. with an intensifying screen. To estimate the amount of target, compare the amount of target detected by visual comparison with hybridization standards of known concentration.

B. Detection of RNA/DNA by Slot-Blot Analysis with Alkaline Phosphatase-Oligonucleotide Conjugate Probes

This example illustrates detection of AGT, ACE, AGTR1, CACNA1C, GPB, EDN1, EDN2, alpha-adducin, haptoglobin, CYP2C9, RGS2, ADRA1a, 11betaHSD2, ADRA1b, ADRA2A, ADRAB1, ADRAB2, REN, APOA, APOB, CETP, LIPC, EDNRB, or ENOS gene(s) mutations by slot-blot detection of DNA with alkaline phosphatase-oligonucleotide conjugate probes, using either a color reagent or a chemiluminescent reagent. The reagents are prepared as follows:

For the color reagent, the following are mixed together, fresh 0.16 mg/ml 5-bromo-4-chloro-3-indolyl phosphate (BCIP), 0.17 mg/ml nitroblue tetrazolium (NBT) in 100 mM NaCl, 100 mM Tris. HCl, 5 mM MgCl.sub.2 and 0.1 mM ZnCl.sub.2, pH 9.5.

Chemiluminescent Reagent:

For the chemiluminescent reagent, the following are mixed together, 250.mu.M 3-adamantyl 4-methoxy 4-(2-phospho)phenyl dioxetane (AMPPD), (Tropix Inc., Bedford, Mass.) in 100 mM diethanolamine-HCl, 1 mM MgCl.sub.2 pH 9.5, or preformulated dioxetane substrate Lumiphos.™. 530 (Lumigen, Inc., Southfield, Mich.).

DNA target (0.01-50 fmol) is immobilized on a nylon membrane as described above. The nylon membrane is incubated in blocking buffer (0.2% I-Block (Tropix, Inc.), 0.5.times. SSC, 0.1% Tween 20) for 30 min. at room temperature with shaking. The filter is then prehybridized in hybridization solution (5.times. SSC, 0.5% BSA, 1% SDS) for 30 minutes at the hybridization temperature (37-60.degree. C.) in a sealable bag using 50-100 .mu.l of hybridization solution per cm of membrane. The solution is removed and briefly washed in warm hybridization buffer. The conjugate probe is then added to give a final concentration of 2-5 nM in fresh hybridization solution and final volume of 50-100 .mu.l/cm.sup.2 of membrane. After incubating for 30 minutes at the hybridization temperature with agitation, the membrane is transferred to a wash tray containing 1.5 ml of preheated wash-1 solution (1.times. SSC, 0.1% SDS)/cm.sup.2 of membrane and agitated at the wash temperature (usually optimum hybridization temperature minus 10.degree. C.) for 10 minutes. Wash-1 solution is removed and this step is repeated once more. Then wash-2 solution (1.times. SSC) added and then agitated at the wash temperature for 10 minutes. Wash-2 solution is removed and immediate detection is done by color.

Detection by color is done by immersing the membrane fully in color reagent, and incubating at 20-37.degree. C. until color development is adequate. When color development is adequate, the development is quenched by washing in water.

For chemiluminescent detection, the following wash steps are performed after the hybridization step (see above). Thus, the membrane is washed for 10 min. with wash-i solution at room temperature, followed by two 3-5 min. washes at 50-60.degree. C. with wash-3 solution (0.5′ SSC, 0.1% SDS). The membrane is then washed once with wash-4 solution (1.times. SSC, 1% Triton X 100) at room temperature for 10 min., followed by a 10 min. wash at room temperature with wash-2 solution. The membrane is then rinsed briefly (.about. 1 min.) with wash-5 solution (50 mM NaHCO.sub.3/1 mM MgCl.sub.2, pH 9.5).

Detection by chemiluminescence is done by immersing the membrane in luminescent reagent, using 25-50 .mu.l solution/cm.sup.2 of membrane. Kodak XAR-5 film (or equivalent; emission maximum is at 477 .mu.m) is exposed in a light-tight cassette for 1-24 hours, and the film developed. EXAMPLE VI

Detection of AGT, ACE, AGTR1, CACNA1C, GPB, EDN1, EDN2, alpha-adducin, haptoglobin, CYP2C9, RGS2, ADRA1a, 11betaHSD2, ADRA1b, ADRA2A, ADRAB1, ADRAB2, REN, APOA, APOB, CETP, LIPC, EDNRB, or ENOS gene(s) Mutations by Amplification and Hybridization

This example illustrates taking a test sample of blood, preparing DNA, amplifying a section of a specific AGT, ACE, AGTR1, CACNA1C, GPB, EDN1, EDN2, alpha-adducin, haptoglobin, CYP2C9, RGS2, ADRA1a, 11betaHSD2, ADRA1b, ADRA2A, ADRAB1, ADRAB2, REN, APOA, APOB, CETP, LIPC, EDNRB, or ENOS gene(s) by polymerase chain reaction (PCR), and detecting the mutation by oligonucleotide hybridization in a dot blot format.

Sample Preparation and Preparing of DNA:

Whole blood is taken from the patient. The blood is lysed, and the DNA prepared for PCR by using procedures described in Example III.

Amplification of Target AGT, ACE, AGTR1, CACNA1C, GPB, EDN1, EDN2, alpha-adducin, haptoglobin, CYP2C9, RGS2, ADRA1a, 11betaHSD2, ADRA1b, ADRA2A, ADRAB1, ADRAB2, REN, APOA, APOB, CETP, LIPC, EDNRB, or ENOS gene(s) by Polymerase Chain Reaction, and Blotting onto Membranes:

The treated DNA from the test sample is amplified using procedures described in Example 1. After amplification, the DNA is denatured, and blotted directly onto prewashed nylon membranes, in multiple aliquots. The membranes are rinsed in 10.times. SSC for five minutes to neutralize the membrane, then rinsed for five minutes in 1.times. SSC. For storage, if any, membranes are air-dried and sealed. In preparation for hybridization, membranes are rinsed in 1.times. SSC, 1% SDS.

Hybridization and Detection:

Hybridization and detection of the amplified genes are accomplished as detailed in Example V.

Although the invention has been described with reference to the disclosed embodiments, those skilled in the art will readily appreciate that the specific examples provided herein are only illustrative of the invention and not limitative thereof. It should be understood that various modifications can be made without departing from the scope of the invention.

### EXAMPLE VII

Synthesis of Antisense Oligonucleotides

Standard manufacturer protocols for solid phase phosphoramidite-based DNA or RNA synthesis using an ABI DNA synthesizer are employed to prepare antisense oligomers. Phosphoroamidite reagent monomers (T, C, A, G, and U) are used as received from the supplier. Applied Biosystems Division/Perkin Elmer, Foster City, Calif. For routine oligomer synthesis, 1 .mu.mole scale syntheses reactions are carried out utilizing THF/I.sub.2/lutidine for oxidation of the phosphoramidite and Beaucage reagent for preparation of the phosphorothioate oligomers. Cleavage from the solid support and deprotection are carried out using ammonium hydroxide under standard conditions. Purification is carried out via reverse phase HPLC and quantification and identification is performed by UV absorption measurements at 260 nm, and mass spectrometry.

### EXAMPLE VIII

Inhibition of Mutant DNA in Cell Culture

Antisense phosphorothioate oligomer complementary to the AGT, ACE, AGTR1, CACNA1C, GPB, EDN1, EDN2, alpha-adducin, haptoglobin, CYP2C9, RGS2, ADRA1a, 11betaHSD2, ADRA1b, ADRA2A, ADRAB1, ADRAB2, REN, APOA, APOB, CETP, LIPC, EDNRB, or ENOS gene(s) gene mutation(s) and thus non-complementary to wild-type AGT, ACE, AGTR1, CACNA1C, GPB, EDN1, EDN2, alpha-adducin, haptoglobin, CYP2C9, RGS2, ADRA1a, 11betaHSD2, ADRA1b, ADRA2A, ADRAB1, ADRAB2, REN, APOA, APOB, CETP, LIPC, EDNRB, or ENOS gene(s) mutant RNA(s), respectively, is added to fresh medium containing Lipofectin.RTM. Gibco BRL (Gaithersburg, Md.) at a concentration of 10 mu.g/ml to make final concentrations of 0.1, 0.33, 1, 3.3, and 10 .mu.M. These are incubated for 15 minutes then applied to the cell culture. The culture is allowed to incubate for 24 hours and the cells are harvested and the DNA isolated and sequenced as in previous examples. Quantitative analysis results shows a decrease in mutant AGT, ACE, AGTR1, CACNA1C, GPB, EDN1, EDN2, alpha-adducin, haptoglobin, CYP2C9, RGS2, ADRA1a, 11betaHSD2, ADRA1b, ADRA2A, ADRAB1, ADRAB2, REN, APOA, APOB, CETP, LIPC, EDNRB, or ENOS gene(s) DNA(s) to a level of less than 1% of total AGT, ACE, AGTR1, CACNA1C, GPB, EDN1, EDN2, alpha-adducin, haptoglobin, CYP2C9, RGS2, ADRA1a, 11betaHSD2, ADRA1b, ADRA2A, ADRAB1, ADRAB2, REN, APOA, APOB, CETP, LIPC, EDNRB, or ENOS gene(s), respectively.

The antisense phosphorothioate oligomer non-complementary to the AGT, ACE, AGTR1, CACNA1C, GPB, EDN1, EDN2, alpha-adducin, haptoglobin, CYP2C9, RGS2, ADRA1a, 11betaHSD2, ADRA1b, ADRA2A, ADRAB1, ADRAB2, REN, APOA, APOB, CETP, LIPC, EDNRB, or ENOS gene(s)gene mutation(s) and non-complementary to wild-type AGT, ACE, AGTR1, CACNA1 C, GPB, EDN1, EDN2, alpha-adducin, haptoglobin, CYP2C9, RGS2, ADRA1a, 11betaHSD2, ADRA1b, ADRA2A, ADRAB1, ADRAB2, REN, APOA, APOB, CETP, LIPC, EDNRB, or ENOS gene(s), respectively is added to fresh medium containing lipofectin at a concentration of 10 .mu.g/mL to make final concentrations of 0. 1, 0.33, 1, 3.3, and 10 .mu.M. These are incubated for 15 minutes then applied to the cell culture. The culture is allowed to incubate for 24 hours and the cells are harvested and the DNA isolated and sequenced as in previous examples. Quantitative analysis results showed no decrease in mutant AGT, ACE, AGTR1, CACNA1C, GPB, EDN1, EDN2, alpha-adducin, haptoglobin, CYP2C9, RGS2, ADRA1a, 11betaHSD2, ADRA1b, ADRA2A, ADRAB1, ADRAB2, REN, APOA, APOB, CETP, LIPC, EDNRB, or ENOS gene(s) DNA, respectively.

### EXAMPLE IX

Inhibition of Mutant DNA in Vivo

Mice are divided into six groups of 10 animals per group. The animals are housed and fed as per standard protocols. To groups 1 to 4 is administered ICV, antisense phosphorothioate oligonucleotide, prepared as described in Example V, complementary to mutant AGT, ACE, AGTR1, CACNA1C, GPB, EDN1, EDN2, alpha-adducin, haptoglobin, CYP2C9, RGS2, ADRA1a, 11betaHSD2, ADRA1b, ADRA2A, ADRAB1, ADRAB2, REN, APOA, APOB, CETP, LIPC, EDNRB, or ENOS gene(s)gene RNA(s), respectively 0.1, 0.33, 1.0 and 3.3 nmol each in 5 .mu.L. To group 5 is administered ICV 1.0 nmol in 5 .mu.L of phosphorothioate oligonucleotide non-complementary to mutant AGT, ACE, AGTR1, CACNA1C, GPB, EDN1, EDN2, alpha-adducin, haptoglobin, CYP2C9, RGS2, ADRA1a, 11betaHSD2, ADRA1b, ADRA2A, ADRAB1, ADRAB2, REN, APOA, APOB, CETP, LIPC, EDNRB, or ENOS gene(s)gene RNA(s) and non-complementary to wild-type AGT, ACE, AGTR1, CACNA1C, GPB, EDN1, EDN2, alpha-adducin, haptoglobin, CYP2C9, RGS2, ADRA1a, 11betaHSD2, ADRA1b, ADRA2A, ADRAB1, ADRAB2, REN, APOA, APOB, CETP, LIPC, EDNRB, or ENOS gene(s) gene RNA(s), respectively. To group 6 is administered ICV vehicle only. Dosing is performed once a day for ten days. The animals are sacrificed and samples of relevant tissue collected. This tissue is treated as previously described and the DNA isolated and quantitatively analyzed as in previous examples. Results show a decrease in mutant ABCB1, ABCB4, COMT, CRHR1, CRHR2, CRHBP, CYP2D6, CYP2D19, DRD2, DRD3, HRT2A, HTR3A, HTR3B, MAOA, MAOB, SLC6A3, and/or or SLC6A4 DNA to a level of less than 1% of total AGT, ACE, AGTR1, CACNA1C, GPB, EDN1, EDN2, alpha-adducin, haptoglobin, CYP2C9, RGS2, ADRA1a, 11betaHSD2, ADRA1b, ADRA2A, ADRAB1, ADRAB2, REN, APOA, APOB, CETP, LIPC, EDNRB, or ENOS gene(s) for the antisense treated group and no decrease for the control group.

Algorithmic Methodology for Determining Relevance of Combinations of Mutations to Hypertension Diagnosis or Prognosis

As was mentioned previously, combinations of mutations might combine in non-linear fashion in determining their effect on diagnosis and prognosis. The present invention demonstrates this as well. A previous example showed that using a trained learning algorithm of neural network and support vector machine type, an average predictability rate of 80% could be achieved in a population that the trained algorithm had never seen before, i.e. an evaluative population.

It is well known to those of ordinary skill in the art that predictive algorithms have three measures of testing, each of increasing validation: how well the algorithm does on data it has learned, called a training population; how well the algorithm does on data that is similar to the original dataset but not trained on, called a testing population; and how well the algorithm does on data it has never seen before, called an evaluation population. What is extremely spectacular about the present invention is its level of predictability in an evaluation population, which indicates its generalizability to a larger population.

It is therefore important to realize that in order to be interpreted into a clinical result that an algorithm must be used to determine the individual contribution each marker makes to the phenotype of interest.

As with identification of the pertinent alleles in the first instance, a algorithm is both (i) selected and (ii) trained to relate (i) identified pre-selected markers and/or characteristics of SNP patterns (as selectively appear in the genomic sequences of each of large number of historical patients) with (ii) the clinical histories of the response of these patients to some particular disease (e.g., breast cancer) in consideration of therapies applied, most commonly drugs. As before, (i) selecting and (ii) training the algorithm to the commonly vast historical clinical data, and to some scores or even hundreds of alleles, is a computationally intensive task normally performed over the period of some hours or days on a supercomputer.

Properly performed—and causal relationships, howsoever complex and permuted, residing somewhere within the data—the resulting (i) selected, and (ii) trained, algorithm will itself be the “synthesis solution”. The algorithm will itself be the expression of what can be known from the data. The later use, and exercise, of the algorithm is only so as to give “answers” for particular questions (i.e., what should be expected from administration of some particular drug) for particular patients (i.e., as are possessed of a particular pattern of markers and/or SNP pattern). Notably, the algorithm can exercised so as to validate its own performance (or lack thereof. The clinical data for the many patients, and patient histories, can be fed into the (selected, trained) algorithm, one patient at a time. Does the algorithm accurately predict what historical data shows to have actually happened? A properly selected and trained algorithm is normally much more accurate in its prognostications (for the useful questions that it may suitably answer) than is any human physician. The physician's judgment ultimately controls, but the “advice” of the algorithm “solution” constitutes a useful adjunct to the physician's judgment in the considerably complex area of relating a patient's therapy to his or her genetic profile.

Without indicating preference for a particular algorithmic technique, one of the preferred embodiments of the present invention is using a neural network to deliver a diagnostic or prognostic prediction using the markers declared previously. In this embodiment, a neural network is used to map the inputs to the outputs. Inputs are selected from using a feature selection algorithm. As above, we note that the construct of a neural network is not crucial to our method. Any mapping procedure between inputs and outputs that produces a measure of goodness of fit for the training data and maximizes it with a standard optimization routine would also suffice.

Once the network is trained, it is ready for use by a clinician. The clinician enters the same network inputs used during training of the network, and the trained network outputs a maximum likelihood estimator for the value of the output given the inputs for the current patient. The clinician or patient can then act on this value. We note that a straightforward extension of our technique could produce an optimum range of output values given the patient's inputs.

In another preferred embodiment of the present invention, an algorithm using a committee network is trained to deliver a diagnostic or prognostic prediction using the markers or combinations thereof declared previously.

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims.

We claim:

1. A method of determining response to the pharmaceutical agent for hypertension, the method comprising: correlating (i) a mutational burden at one or more nucleotide positions in the AGT, ACE, AGTR1, CACNA1C, GPB, EDN1, EDN2, alpha-adducin, haptoglobin, CYP2C9, RGS2, ADRA1a, 11betaHSD2, ADRA1b, ADRA2A, ADRAB1, ADRAB2, REN, APOA, APOB, CETP, LIPC, EDNRB, or ENOS gene(s) in a sample from the subject with (ii) the mutational burden at one or more corresponding nucleotide positions in a control sample with known response outcome, and therefrom identifying the probability of response to said pharmaceutical agent.

2. A method according to claim 1 wherein the mutational burden relates to a mutation in the CACNA1C gene at nucleotide position given by the RS # 2238032, 2239050, or 769087, further described by the genetic position descriptor chr12:2092993-2092993, chr12:2317675-2317675, or chr12:2214905-2214905, respectively; or combinations thereof, and the pharmaceutical agent is a calcium-channel blocker.

3. A method according to claim 2 wherein the mutational burden is comprised of at least one mutation in linkage disequilibrium with the genetic variants according to claim 2.

4. A method according to claim 1 wherein the mutational burden relates to a mutation in the AGTR1 gene at nucleotide position given by the RS # 5186 further described by the genetic position descriptor chr3:149942686; a mutation in the ADRB2 gene at nucleotide position given by the RS # 1042720 further described by the genetic position descriptor chr5:148187826; and mutations in the ADRB1 gene at nucleotide position given by the RS # 2183378 and RS # 2050393, further described by the genetic position descriptors chr10:115798882 and chr10:115804942, respectively; or combinations thereof, and the pharmaceutical agent is a beta- blocker.

5. A method according to claim 4 wherein the mutational burden is comprised of at least one mutation in linkage disequilibrium with the genetic variants according to claim 4.

6. A method according to claim 1, wherein said correlating step comprising: a) determining the sequence of one or more of the genes AGT, ACE, AGTR1, CACNA1C, GPB, EDN1, EDN2, alpha-adducin, haptoglobin, CYP2C9, RGS2, ADRA1a, 11betaHSD2, ADRA1b, ADRA2A, ADRAB1, ADRAB2, REN, APOA, APOB, CETP, LIPC, EDNRB, or ENOS from humans known to be responsive or non-responsive to anti- hypertension medications; b) comparing said sequence to that of the corresponding wildtype AGT, ACE, AGTR1, CACNA1C, GPB, EDN1, EDN2, alpha-adducin, haptoglobin, CYP2C9, RGS2, ADRA1a, 11betaHSD2, ADRA1b, ADRA2A, ADRAB1, ADRAB2, REN, APOA, APOB, CETP, LIPC, EDNRB, or ENOS gene(s); and c) training an algorithm to identify patterns of identifying mutations which correlate with the response or non-response to anti-hypertensive medications, respectively.

7. The method according to claim 6, where training said algorithm residing on a computer on characteristic mutations comprises the steps of obtaining numerous examples of (i) said SNP pattern genomic data, and (ii) historical clinical results corresponding to this genomic data;
constructing a algorithm suitable to map (i) said SNP pattern genomic data as inputs to the algorithm to (ii) the historical clinical results as outputs of the algorithm;
exercising the constructed algorithm to so map (i) the said SNP pattern genomic data as inputs to (ii) the historical clinical results as outputs; and
conducting an automated procedure to vary the mapping function, inputs to outputs, of the constructed and exercised algorithm in order that, by minimizing an error measure of the mapping function, a more optimal algorithm mapping architecture is realized;
wherein realization of the more optimal algorithm mapping architecture, also known as feature selection, means that any irrelevant inputs are effectively excised, meaning that the more optimally mapping algorithm will substantially ignore input alleles and/or said SNP pattern genomic data that is irrelevant to output clinical results; and
wherein realization of the more optimal algorithm mapping architecture, also known as feature selection, also means that any relevant inputs are effectively identified, making that the more optimally mapping algorithm will serve to identify, and use, those input alleles and/or SNP pattern genomic data that is relevant, in combination, to output clinical results.

8. The method according to claim 7, where the constructed algorithm is drawn from the group consisting essentially of: linear or nonlinear regression algorithms; linear or nonlinear classification algorithms; ANOVA; neural network algorithms; genetic algorithms; support vector machines algorithms; hierarchical analysis or clustering algorithms; hierarchical algorithms using decision trees; kernel based machine algorithms such as kernel partial least squares algorithms, kernel matching pursuit algorithms, kernel fisher discriminate analysis algorithms, or kernel principal components analysis algorithms; Bayesian probability function algorithms; Markov Blanket algorithms; a plurality of algorithms arranged in a committee network; and forward floating search or backward floating search algorithms.

9. The method according to claim 7, where the feature selection process employs an algorithm drawn from the group consisting essentially of: linear or nonlinear regression algorithms; linear or nonlinear classification algorithms; ANOVA; neural network algorithms; genetic algorithms; support vector machines algorithms; hierarchical analysis or clustering algorithms; hierarchical algorithms using decision trees; kernel based machine algorithms such as kernel partial least squares algorithms, kernel matching pursuit algorithms, kernel fisher discriminate analysis algorithms, or kernel principal components analysis algorithms; Bayesian probability function algorithms; Markov Blanket algorithms; recursive feature elimination or entropy-based recursive feature elimination algorithms; a plurality of algorithms arranged in a committee network; and forward floating search or backward floating search algorithms.

10. The method according to claim 7, wherein a tree algorithm is trained to reproduce the performance of another machine-learning classifier or regressor by enumerating the input space of said classifier or regressor to form a plurality of training examples sufficient (1) to span the input space of said classifier or regressor and (2) train the tree to emulate the performance of said classifier or regressor.

11. The method according to claim 6 where the anti-hypertensive medication belongs to the class known as angiotensin converting enzyme inhibitors, calcium channel blockers, or beta-adrenergic receptor blockers.

12. The method according to claim 6 where the anti-hypertensive medication is the molecules captopril, benazepril, enalapril, enalaprilat, fosinopril, lisinopril, quinapril, ramipril, and trandolapril or others with similar molecular mechanisms; the molecules nifedipine, verapamil, nicardipine,diltiazem, isradipine, amlodipine, nimodipine, felodipine, nisoldipine, bepridil or others with similar molecular mechanisms; and the molecules atenolol, metoprolol, propranolol, timolol, nadolol, acebutolol, pindolol, sotalol, labetalol, oxprenolol or others with similar molecular mechanisms.

13. The method of claim 1 wherein at least one mutation is a silent mutation, missense mutation, or combination thereof.

14. A method according to claim 1, wherein said sample is selected from the group consisting of a blood sample, a serum sample, and a plasma sample.

15. A method according to any one of claims 1 wherein the presence of said mutation is detected by a technique that is selected from the group of techniques consisting of hybridization with oligonucleotide probes, a ligation reaction, a polymerase chain reaction and single nucleotide primer-guided extension assays, and variations thereof.

16. A method according to claim 1, wherein said correlating step comprises comparing said mutational burden to a second mutational burden measured in a second sample obtained from said patient, whereby, when said second mutational burden is of the type correlated by one or more of algorithm(s) drawn from the group consisting essentially of linear or nonlinear regression algorithms; linear or nonlinear classification algorithms; ANOVA; neural network algorithms; genetic algorithms; support vector machines algorithms; hierarchical analysis or clustering algorithms; hierarchical algorithms using decision trees; kernel based machine algorithms such as kernel partial least squares algorithms, kernel matching pursuit algorithms, kernel fisher discriminate analysis algorithms, or kernel principal components analysis algorithms; Bayesian probability function algorithms; Markov Blanket algorithms; recursive feature elimination or entropy-based recursive feature elimination algorithms; a plurality of algorithms arranged in a committee network; and forward floating search or backward floating search algorithms than said second mutational burden, said patient is diagnosed as being responsive or resistant to anti-hypertensive therapy.

17. A method according to claim 16, wherein said second sample is obtained prior to treatment with an anti-hypertensive medication.

18. A method for detecting the presence or risk of developing hypertension in a human, said method comprising: determining the presence in a biological sample from a human of a nucleic acid sequence having a mutational burden according to claim 1 at one or more nucleotide positions in a sequence region corresponding to a wildtype genomic DNA sequence, wherein the mutational burden correlates with the presence of or risk of developing hypertension.

19. A method for evaluating a compound for use in diagnosis or treatment of hypertension, said method comprising: a) contacting a predetermined quantity of said compound with cultured cybrid cells or animal model having genomic DNA originating from a neuronal rho or human embryonic immortal kidney cell line and from tissue of a human having a disorder that is associated with severe hypertension and the mutational burden according to claim 2; b) measuring a phenotypic trait in said cybrid cells or animal model that correlates with the presence of said mutational burden and that is not present in cultured cybrid cells or animal model having genomic DNA originating from a neuronal rho cell line and genomic DNA originating from tissue of a human free of a disorder that is associated with severe hypertension; and c) correlating a change in the phenotypic trait with effectiveness of the compound.

20. A method according to claim 19 where the phenotypic trait is blockade of of at least one cascade in the renin-angiotensin-aldosterone biochemical pathway, calcium channel pathway, or beta-adrenergic pathway.

21. A method according to claim 19 where the correlating step is made in accordance with an algorithm drawn from the group consisting essentially of: linear or nonlinear regression algorithms; linear or nonlinear classification algorithms; ANOVA; neural network algorithms; genetic algorithms; support vector machines algorithms; hierarchical analysis or clustering algorithms; hierarchical algorithms using decision trees; kernel based machine algorithms such as kernel partial least squares algorithms, kernel matching pursuit algorithms, kernel fisher discriminate analysis algorithms, or kernel principal components analysis algorithms; Bayesian probability function algorithms; Markov Blanket algorithms; recursive feature elimination or entropy-based recursive feature elimination algorithms; a plurality of algorithms arranged in a committee network; and forward floating search or backward floating search algorithms.

22. A method for diagnosing treatment-resistant hypertension, said method comprising: determining the presence in a biological sample from a human of a nucleic acid sequence having a mutational burden according to claim 2 at one or more nucleotide positions in a sequence region corresponding to a wildtype genomic DNA sequence, wherein the mutational burden correlates with the lack of response to a hypertension medication.

23. A method according to claim 22 where the correlating step is made in accordance with an algorithm drawn from the group consisting essentially of: linear or nonlinear regression algorithms; linear or nonlinear classification algorithms; ANOVA; neural network algorithms; genetic algorithms; support vector machines algorithms; hierarchical analysis or clustering algorithms; hierarchical algorithms using decision trees; kernel based machine algorithms such as kernel partial least squares algorithms, kernel matching pursuit algorithms, kernel fisher discriminate analysis algorithms, or kernel principal components analysis algorithms; Bayesian probability function algorithms; Markov Blanket algorithms; recursive feature elimination or entropy-based recursive feature elimination algorithms; a plurality of algorithms arranged in a committee network; and forward floating search or backward floating search algorithms.

24. A method according to claim 22, wherein said specific marker for treatment-resistant hypertension is selected from the group of genes consisting of AGT, ACE, AGTR1, CACNA1C, GPB, EDN1, EDN2, ALPHA-ADDUCIN, HAPTOGLOBIN, CYP2C9, RGS2, ADRA1A, 11BETAHSD2, ADRA1B, ADRA2A, ADRAB1, ADRAB2, REN, APOA, APOB, CETP, LIPC, EDNRB, OR ENOS.

25. A therapeutic composition comprising antisense or small interfering RNA sequences which are specific to mutant genes according to claim 1 or mutant messenger RNA transcribed therefrom, said antisense or small interfering RNA sequences adapted to bind to and inhibit transcription or translation of said target genes according to claim 1 without preventing transcription or translation of wild-type genes of the same type.

26. The therapeutic composition of claim 25, wherein hypertension is treated and wherein said mutant genes are selected from the group: AGT, ACE, AGTR1, CACNAC1, GPB, EDN1, EDN2, ALPHA-ADDUCIN, HAPTOGLOBIN, CYP2C9, RGS2, ADRA1A, 11BETAHSD2, ADRA1B, ADRA2A, ADRAB1, ADRAB2, REN, APOA, APOB, CETP, LIPC, EDNRB, OR ENOS.

27. A kit comprising devices and reagents and a computer algorithm for measuring one or more mutational burdens of a patient and determining the diagnosis or prognosis in that patient for hypertension.

28. The method of claim 27 when the mutational burden is a mutation in the CACNA1C gene at nucleotide position given by the RS # 2238032, 2239050, or 769087, further described by the genetic position descriptor chr12:2092993-2092993, chr12:2317675-2317675, or chr12:2214905-2214905, respectively; a mutation in the AGTR1 gene at nucleotide position given by the RS # 5186 further described by the genetic position descriptor chr3:149942686; a mutation in the ADRB2 gene at nucleotide position given by the RS # 1042720 further described by the genetic position descriptor chr5:148187826; and mutations in the ADRB1 gene at nucleotide position given by the RS # 2183378 and RS # 2050393, further described by the genetic position descriptors chr10:115798882 and chr10:115804942, respectively; a mutation in linkage disequilibrium with the above genetic variants; or combinations thereof.

29. The method of claim 27 when the determination of diagnostic or prognostic outcome is made in accordance with an algorithm drawn from the group consisting essentially of: linear or nonlinear regression algorithms; linear or nonlinear classification algorithms; ANOVA; neural network algorithms; genetic algorithms; support vector machines algorithms; hierarchical analysis or clustering algorithms; hierarchical algorithms using decision trees; kernel based machine algorithms such as kernel partial least squares algorithms, kernel matching pursuit algorithms, kernel fisher discriminate analysis algorithms, or kernel principal components analysis algorithms; Bayesian probability function algorithms; Markov Blanket algorithms; recursive feature elimination or entropy-based recursive feature elimination algorithms; a plurality of algorithms arranged in a committee network; and forward floating search or backward floating search algorithms.

30. The method of claim 27 when the prognostic outcome is that of response to ACE anti-hypertension medication, a calcium channel blocker anti-hypertension medication, or a beta-blocker anti-hypertension medication.

31. The method of claim 30 the determination of diagnostic or prognostic outcome is made in accordance with an algorithm drawn from the group consisting essentially of: linear or nonlinear regression algorithms; linear or nonlinear classification algorithms; ANOVA; neural network algorithms; genetic algorithms; support vector machines algorithms; hierarchical analysis or clustering algorithms; hierarchical algorithms using decision trees; kernel based machine algorithms such as kernel partial least squares algorithms, kernel matching pursuit algorithms, kernel fisher discriminate analysis algorithms, or kernel principal components analysis algorithms; Bayesian probability function algorithms; Markov Blanket algorithms; recursive feature elimination or entropy-based recursive feature elimination algorithms; a plurality of algorithms arranged in a committee network; and forward floating search or backward floating search algorithms.

32. The method of claim 27 when the diagnostic outcome is that of treatment-resistant hypertension.

33. The method of claim 32 when the determination of diagnostic or prognostic outcome is made in accordance with an algorithm drawn from the group consisting essentially of: linear or nonlinear regression algorithms; linear or nonlinear classification algorithms; ANOVA; neural network algorithms; genetic algorithms; support vector machines algorithms; hierarchical analysis or clustering algorithms; hierarchical algorithms using decision trees; kernel based machine algorithms such as kernel partial least squares algorithms, kernel matching pursuit algorithms, kernel fisher discriminate analysis algorithms, or kernel principal components analysis algorithms; Bayesian probability function algorithms; Markov Blanket algorithms; recursive feature elimination or entropy-based recursive feature elimination algorithms; a plurality of algorithms arranged in a committee network; and forward floating search or backward floating search algorithms.

34. The method of claim 27 when the prognostic outcome is that of response to the molecules captopril, benazepril, enalapril, enalaprilat, fosinopril, lisinopril, quinapril, ramipril, and trandolapril or others with similar molecular mechanisms; the molecules nifedipine, verapamil, nicardipine,diltiazem, isradipine, amlodipine, nimodipine, felodipine, nisoldipine, bepridil or others with similar molecular mechanisms; and the molecules atenolol, metoprolol, propranolol, timolol, nadolol, acebutolol, pindolol, sotalol, labetalol, oxprenolol or others with similar molecular mechanisms.

35. The method of claim 34 when the determination of diagnostic or prognostic outcome is made in accordance with an algorithm drawn from the group consisting essentially of: linear or nonlinear regression algorithms; linear or nonlinear classification algorithms; ANOVA; neural network algorithms; genetic algorithms; support vector machines algorithms; hierarchical analysis or clustering algorithms; hierarchical algorithms using decision trees; kernel based machine algorithms such as kernel partial least squares algorithms, kernel matching pursuit algorithms, kernel fisher discriminate analysis algorithms, or kernel principal components analysis algorithms; Bayesian probability function algorithms; Markov Blanket algorithms; recursive feature elimination or entropy-based recursive feature elimination algorithms; a plurality of algorithms arranged in a committee network; and forward floating search or backward floating search algorithms.

36. The method of claim 27 when the diagnostic outcome is that of determining risk of hypertension.

37. The method of claim 36 when the determination of diagnostic or prognostic outcome is made in accordance with an algorithm drawn from the group consisting essentially of: linear or nonlinear regression algorithms; linear or nonlinear classification algorithms; ANOVA; neural network algorithms; genetic algorithms; support vector machines algorithms; hierarchical analysis or clustering algorithms; hierarchical algorithms using decision trees; kernel based machine algorithms such as kernel partial least squares algorithms, kernel matching pursuit algorithms, kernel fisher discriminate analysis algorithms, or kernel principal components analysis algorithms; Bayesian probability function algorithms; Markov Blanket algorithms; recursive feature elimination or entropy-based recursive feature elimination algorithms; a plurality of algorithms arranged in a committee network; and forward floating search or backward floating search algorithms.
